(19) United States
(12) Patent Application Publication
(10) Pub. No.: US 2026/0184793 A1
(43) Pub. Date: Jul. 2, 2026

(54) ANTI-PD-L1 NANOBODIES

(71) Applicant: TJ BIOPHARMA (SHANGHAI) CO., LTD., Shanghai (CN)

(72) Inventors: Jian LI, Shanghai (CN); Hui YANG, Shanghai (CN)

(21) Appl. No.: 19/130,109

(22) PCT Filed: Nov. 15, 2023
(86) PCT No.: PCT/CN2023/131749

§ 371 Date: May 14, 2025

(30) Foreign Application Priority Data
Nov. 15, 2022 (WO) PCT/CN2022/131894

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/31; C07K 2317/565; C07K 2317/567; C07K 2317/569; C07K 2317/92

(57) ABSTRACT

Provided are affinity-maturated single domain anti-PD-L1 antibodies and polypeptides, such as multispecific antibodies and chimeric antigen receptors, that include these single domain antibodies. These antibodies, including their humanized counterparts, exhibited superior activities and are suitable for use in various multispecific antibody formats. Methods of using the antibodies or polypeptides for treating and diagnosing diseases such as cancer and infectious diseases are also provided.

### BACKGROUND

A single domain antibody (sdAb), also known as a nanobody, is an antibody fragment consisting of a single monomeric variable antibody domain. Like a whole antibody, it is able to bind selectively to a specific antigen. With a molecular weight of only 12-15 kDa, single domain antibodies are much smaller than common antibodies (150-160 kDa). Single domain antibodies, given their small sizes and one-chain nature, can be particularly suitable for inclusion as a fragment in other proteins, such as bispecific antibodies.

Antibodies specific to programmed death-ligand 1 (PD-L1), also known as cluster of differentiation 274 (CD274) or B7 homolog 1 (B7-H1), are being used for cancer treatments and in other clinical applications. PD-L1 is a 40 kDa type 1 transmembrane protein believed to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2.

In addition to the treatment of cancers, PD-L1 inhibition has also shown promises in treating infectious diseases. In a mouse model of intracellular infection, L. monocytogenes induced PD-L1 protein expression in T cells, NK cells, and macrophages. PD-L1 blockade (e.g., using blocking antibodies) resulted in increased mortality for infected mice. PD-L1 blockade reduced TNFα and nitric oxide production by macrophages, reduced granzyme B production by NK cells, and decreased proliferation of L. monocytogenes antigen-specific CD8 T cells (but not CD4 T cells). This evidence suggests that PD-L1 acts as a positive costimulatory molecule in intracellular infection.

### SUMMARY

The present disclosure provides new single domain antibodies and affinity maturated counterparts targeting the human PD-L1 protein. These single domain antibodies, despite their small sizes, exhibited superior binding affinity and biological functions. When included in various different formats of multispecific antibodies, some of the resulting multispecific antibodies exhibited excellent properties.

One embodiment of the present disclosure provides a single domain antibody or a polypeptide comprising the single domain antibody, wherein the single domain antibody has binding specificity to the human PD-L1 protein and comprises a complementarity determining region 1 (CDR1), a CDR2 and a CDR3, wherein:
(1) the CDR1 comprises the amino acid sequence of SEQ ID NO:54 or 95;
   the CDR2 comprises the amino acid sequence of SEQ ID NO:55 or 96; and
   the CDR3 comprises the amino acid sequence of SEQ ID NO:56, 97, 98, 99, 100, 101, 102 or 103, wherein the CDR1, CDR2 and CDR3 do not comprise the sequences of SEQ ID NO:54-56, respectively; or
(2) the CDR1 comprises the amino acid sequence of SEQ ID NO:57 or 90;
   the CDR2 comprises the amino acid sequence of SEQ ID NO:58 or 91; and
   the CDR3 comprises the amino acid sequence of SEQ ID NO:59, 92, 93 or 94, wherein the CDR1, CDR2 and CDR3 do not comprise the sequences of SEQ ID NO:57-59, respectively.

In some embodiments, the CDR1, CDR2 and CDR3 comprise, respectively,
(1) SEQ ID NO:90, 58, and 59;
(2) SEQ ID NO:57, 91, and 59;
(3) SEQ ID NO:57, 58, and 92;
(4) SEQ ID NO:57, 58, and 93;
(5) SEQ ID NO:90, 58, and 92;
(6) SEQ ID NO:90, 58, and 93;
(7) SEQ ID NO:57, 91, and 92;
(8) SEQ ID NO:57, 91, and 93;
(9) SEQ ID NO:57, 58, and 94;
(10) SEQ ID NO:90, 91, and 94;
(11) SEQ ID NO:90, 91, and 92; or
(12) SEQ ID NO:90, 91, and 93.

In some embodiments, the antibody or polypeptide comprises an amino acid sequence having at least 85% sequence identity to SEQ ID NO:53. In some embodiments, the antibody or polypeptide comprises the framework regions of SEQ ID NO:53.

In some embodiments, the CDR1, CDR2 and CDR3 comprise, respectively, SEQ ID NO: 57, 91, and 93. In some embodiments, the antibody or polypeptide comprises the amino acid sequence of SEQ ID NO:67.

In some embodiments, the CDR1, CDR2 and CDR3 comprise, respectively, SEQ ID NO: 90, 91, and 92. In some embodiments, the antibody or polypeptide comprises the amino acid sequence of SEQ ID NO:70.

In some embodiments, the CDR1, CDR2 and CDR3 comprise, respectively,
(13) SEQ ID NO:95, 55, and 56;
(14) SEQ ID NO:54, 96, and 56;
(15) SEQ ID NO:54, 55, and 97;
(16) SEQ ID NO:54, 55, and 98;
(17) SEQ ID NO:54, 55, and 99;
(18) SEQ ID NO:95, 55, and 97;
(19) SEQ ID NO:95, 55, and 98;
(20) SEQ ID NO:95, 55, and 99;
(21) SEQ ID NO:54, 96, and 97;
(22) SEQ ID NO:54, 96, and 98;
(23) SEQ ID NO:54, 96, and 99;
(24) SEQ ID NO:95, 96, and 97;
(25) SEQ ID NO:95, 96, and 98;
(26) SEQ ID NO:95, 96, and 99;
(27) SEQ ID NO:95, 96, and 100;
(28) SEQ ID NO:54, 55, and 101;
(29) SEQ ID NO:54, 55, and 102; or
(30) SEQ ID NO:54, 55, and 103.

In some embodiments, the antibody or polypeptide comprises an amino acid sequence having at least 85% sequence identity to SEQ ID NO:42. In some embodiments, the antibody or polypeptide comprises the framework regions of SEQ ID NO:42.

In some embodiments, the CDR1, CDR2 and CDR3 comprise, respectively, SEQ ID NO: 95, 96, and 100. In some embodiments, the antibody or polypeptide comprises the amino acid sequence of SEQ ID NO:86.

Also provided, in one embodiment, is a multispecific antibody comprising the antibody of the present disclosure and a second antibody or antigen-binding fragment having binding specificity to a target antigen that is not PD-L1. In some embodiments, the target antigen is a tumor-associated antigen. In some embodiments, the second antibody is a full-size Fab antibody.

In another aspect, provided herein is a polynucleotide encoding the antibody or polypeptide of the present application, or the multispecific antibody of the present application.

In another aspect, provided herein is a vector comprising the polynucleotide of the present application.

In another aspect, provided is a cell comprising the polynucleotide or the vector of the present application.

In another aspect, provided herein is a composition comprising: (1) the antibody or polypeptide, the multispecific antibody, or the polynucleotide of the present application, and (2) a pharmaceutically acceptable carrier.

In another aspect, provided herein is a method of treating cancer in a patient in need thereof, comprising administering to the patient an effective amount of the antibody or polypeptide, the multispecific antibody, or the polynucleotide of the present application. In another aspect, provided herein is use of the antibody or polypeptide, the multispecific antibody, or the polynucleotide of the present application for the preparation of a medicament for treating cancer. In some embodiments, the cancer is a solid tumor. In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

### BRIEF DESCRIPTION OF THE DRAWINGS

FIG. 1 shows the results of ELISA binding of the affinity matured nanobodies.

FIG. 2 shows the results of cell-based PD-L1 binding on PDL-1 overexpressed cells.

FIG. 3 shows the results of PD-L1 ELISA blocking assay.

FIG. 4 shows the results of PD-1/PD-L1 functional reporter assay.

FIG. 5 shows the results of PDL-1 nano mediated human IL-2 release in Mixed Lymphocyte Reaction (MLR) assay.

FIG. 6 shows the in vivo anti-tumor effect of the affinity matured nanobodies.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Single Domain Anti-PD-L1 Antibodies

The present disclosure provides single chain anti-PD-L1 antibodies and their humanized versions with high affinity to the human PD-L1 protein. Some of the top antibodies were further subjected to affinity maturation, and some of the affinity maturated candidates exhibited superior performance. These new antibodies exhibited potent binding and inhibitory activities and are useful for therapeutic and diagnostics uses. Also importantly, when incorporated as one of the targeting units in a variety of different formats of multispecific antibodies, certain resulting multispecific antibodies exhibited outstanding properties, establishing the additional utility of these single domain anti-PD-L1 antibodies.

Accordingly, in one embodiment of the present disclosure, provided are single domain antibodies and polypeptides that include such a single domain antibody. In some embodiments, the polypeptide is a bispecific antibody, a tri-specific antibody, or a multi-specific antibody.

One embodiment of the present disclosure provides a single domain antibody or a polypeptide comprising the single domain antibody, wherein the single domain antibody has binding specificity to the human PD-L1 protein and includes a complementarity determining region 1 (CDR1), a CDR2 and a CDR3.

In some embodiments, an affinity maturated single domain anti-PD-L1 antibody is derived from parental antibody 112-VH47 (SEQ ID NO:53). Accordingly, in some embodiments, the CDR1 includes the amino acid sequence of SEQ ID NO:54 or 95; the CDR2 includes the amino acid sequence of SEQ ID NO:55 or 96; and the CDR3 includes the amino acid sequence of SEQ ID NO:56, 97, 98, 99, 100, 101, 102 or 103, wherein the CDR1, CDR2 and CDR3 do not include the sequences of SEQ ID NO:54-56, respectively.

In some embodiments, the CDR1, CDR2 and CDR3 include, respectively, SEQ ID NO: 90, 58, and 59. In some embodiments, the CDR1, CDR2 and CDR3 include, respectively, SEQ ID NO:57, 91, and 59. In some embodiments, the CDR1, CDR2 and CDR3 include, respectively, SEQ ID NO:57, 58, and 92. In some embodiments, the CDR1, CDR2 and CDR3 include, respectively, SEQ ID NO:57, 58, and 93. In some embodiments, the CDR1, CDR2 and CDR3 include, respectively, SEQ ID NO:90, 58, and 92. In some embodiments, the CDR1, CDR2 and CDR3 include, respectively, SEQ ID NO:90, 58, and 93. In some embodiments, the CDR1, CDR2 and CDR3 include, respectively, SEQ ID NO:57, 91, and 92. In some embodiments, the CDR1, CDR2 and CDR3 include, respectively, SEQ ID NO:57, 91, and 93. In some embodiments, the CDR1, CDR2 and CDR3 include, respectively, SEQ ID NO:57, 58, and 94. In some embodiments, the CDR1, CDR2 and CDR3 include, respectively, SEQ ID NO: 90, 91, and 94. In some embodiments, the CDR1, CDR2 and CDR3 include, respectively, SEQ ID NO:90, 91, and 92. In some embodiments, the CDR1, CDR2 and CDR3 include, respectively, SEQ ID NO:90, 91, and 93. In some embodiments, the CDR1, CDR2 and CDR3 include, respectively, SEQ ID NO:95, 55, and 56.

In some embodiments, the antibody or polypeptide, which includes the above CDRs, includes an amino acid sequence having at least 70%, 75%, 80%, 85%, 90%, 95%, 97%, 98% or 99% sequence identity to SEQ ID NO:53. In some embodiments, the antibody or polypeptide includes the framework regions of SEQ ID NO:53.

In some embodiments, the CDR1, CDR2 and CDR3 include, respectively, SEQ ID NO: 57, 91, and 93. In some embodiments, the antibody or polypeptide includes the amino acid sequence of SEQ ID NO:67.

In some embodiments, the CDR1, CDR2 and CDR3 include, respectively, SEQ ID NO: 90, 91, and 92. In some embodiments, the antibody or polypeptide includes the amino acid sequence of SEQ ID NO:70.

In some embodiments, an affinity maturated single domain anti-PD-L1 antibody is derived from parental antibody 93_VH-6 (SEQ ID NO:42). Accordingly, in some embodiments, the CDR1 includes the amino acid sequence of SEQ ID NO:57 or 90; the CDR2 includes the amino acid sequence of SEQ ID NO:58 or 91; and the CDR3 includes the amino acid sequence of SEQ ID NO:59, 92, 93 or 94, wherein the CDR1, CDR2 and CDR3 do not include the sequences of SEQ ID NO:57-59, respectively.

In some embodiments, the CDR1, CDR2 and CDR3 include, respectively, SEQ ID NO: 54, 96, and 56. In some embodiments, the CDR1, CDR2 and CDR3 include, respectively, SEQ ID NO:54, 55, and 97. In some embodiments, the CDR1, CDR2 and CDR3 include, respectively, SEQ ID NO:54, 55, and 98. In some embodiments, the CDR1, CDR2 and CDR3 include, respectively, SEQ ID NO:54, 55, and 99. In some embodiments, the CDR1, CDR2 and CDR3 include, respectively, SEQ ID NO:95, 55, and 97. In some embodiments, the CDR1, CDR2 and CDR3 include, respectively, SEQ ID NO:95, 55, and 98. In some embodiments, the CDR1, CDR2 and CDR3 include, respectively, SEQ ID NO:95, 55, and 99. In some embodiments, the CDR1, CDR2 and CDR3 include, respectively, SEQ ID NO:54, 96, and 97. In some embodiments, the CDR1, CDR2 and CDR3 include, respectively, SEQ ID NO:54, 96, and 98. In some embodiments, the CDR1, CDR2 and CDR3 include, respectively, SEQ ID NO: 54, 96, and 99. In some embodiments, the CDR1, CDR2 and CDR3 include, respectively, SEQ ID NO:95, 96, and 97. In some embodiments, the CDR1, CDR2 and CDR3 include, respectively, SEQ ID NO:95, 96, and 98. In some embodiments, the CDR1, CDR2 and CDR3 include, respectively, SEQ ID NO:95, 96, and 99. In some embodiments, the CDR1, CDR2 and CDR3 include, respectively, SEQ ID NO:95, 96, and 100. In some embodiments, the CDR1, CDR2 and CDR3 include, respectively, SEQ ID NO:54, 55, and 101. In some embodiments, the CDR1, CDR2 and CDR3 include, respectively, SEQ ID NO:54, 55, and 102. In some embodiments, the CDR1, CDR2 and CDR3 include, respectively, SEQ ID NO:54, 55, and 103.

In some embodiments, the antibody or polypeptide, which includes the above CDRs, includes an amino acid sequence having at least 70%, 75%, 80%, 85%, 90%, 95%, 97%, 98% or 99% sequence identity to SEQ ID NO:42. In some embodiments, the antibody or polypeptide includes the framework regions of SEQ ID NO:42.

In some embodiments, the CDR1, CDR2 and CDR3 include, respectively, SEQ ID NO: 95, 96, and 100. In some embodiments, the antibody or polypeptide includes the amino acid sequence of SEQ ID NO:86.

Also provided, in some embodiments, are anti-PD-L1 antibodies and antigen binding fragments that compete with any of the antibodies disclosed herein in binding to human PD-L1. Also provided, in some embodiments, are anti-PD-L1 antibodies and antigen binding fragments that bind to the same epitope as any of the antibodies disclosed herein. Also provided, in some embodiments, are anti-PD-L1 antibodies and antigen binding fragments that included the VH CDR1, CDR2, and CDR3 and VL CDR1, CDR2 and CDR3 of the antibodies disclosed herein.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

### Multispecific Antibodies

Also provided are bispecific and multispecific antibodies that include one, two, three or four units of the single domain anti-PD-L1 antibody as disclosed herein, and one or more other specificities (not PD-L1).

In some embodiments, the second specificity is to a tumor associated antigen (TAA) or immune checkpoint protein. Non-limiting examples of tumor associated antigen include EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGFIR, EPHA3, TRAILR1, TRAILR2, RANKL, FAP, Tenascin, and Claudin 18.2.

Non-limiting examples immune checkpoint protein include PD-1, CTLA-4, LAG-3, CD28, CD122, 4-1BB, TIM3, OX-40, OX40L, CD40, CD40L, LIGHT, ICOS, ICOSL, GITR, GITRL, CD27, VISTA, B7H3, B7H4, HEVM, BTLA, KIR, and CD47.

The multispecific antibodies may include constant regions from any IgG types, such as IgG1 and IgG4.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Cancer Treatment

As described herein, the antibodies, multispecific antibodies, polypeptides, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. PD-L1 is reported to be overexpressed in tumor cells. Tumor-derived PD-L1 can bind to PD-1 on immune cells thereby limiting antitumor T-cell immunity. Results with small molecule inhibitors, or monoclonal antibodies targeting PD-L1 in murine tumor models, indicate that targeted PD-L1 therapy is an important alternative and realistic approach to effective control of tumor growth. As demonstrated in the experimental examples, the anti-PD-L1 antibodies activated the adaptive immune response machinery, which can lead to improved survival in cancer patients.

Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient expresses, over-express, or is induced to express PD-L1. Induction of PD-L1 expression, for instance, can be done by administration of a tumor vaccine or radiotherapy.

Tumors that express the PD-L1 protein include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, oesophageal cancer, ovarian cancer, renal cancer, and small cell lung cancer. Accordingly, the presently disclosed antibodies can be used for treating any one or more such cancers.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1. Generation of Alpaca Single Domain Antibodies Against Human PD-L1

This example shows how anti-human-PD-L1 single domain antibodies were generated using immunization of alpaca followed by phage library construction and selection.

Antigen: Recombinant human PD-L1/hFc fusion proteins were used as the immunogen to raise anti-human PD-L1 antibodies. A fusion protein comprising the entire extracellular region of human PD-L1 fused to a human immunoglobulin Fc domain was used as the immunogen.

### Immunization

Alpacas were first subcutaneously (SC) immunized with a 1:1 mixture of 600 μg mouse PD-L1 and complete Freud's adjuvant on day 0 and immunized with 250 μg mouse PD-L1 with incomplete Freud's adjuvant on day 21 and 250 μg human PD-L1 with incomplete Freud's adjuvant on day 42. The immune response was monitored by measuring titers for anti-PD-L1 binding.

### Library Construction and Screening

Alpaca PBMCs were collected, and an antibody phage display library was generated by RNA isolation, cDNA reverse transcription, PCR amplification and cloning into a phage display vector. The library was then subjected for one round of liquid phase panning and one round of solid phase panning. In general, the libraries were incubated in biotinylated PD-L1-coated immunotubes or beads. Unbound phages were removed by washing with PBST for 5-20 times. For each selection, three rounds of panning were performed in total.

The binder sequences were amplified from antigen-binding positive phages by PCR and confirmed by DNA sequencing. Sequences of the unique antibodies and their CDR regions are provided in the table below.

TABLE 1
Antibody Sequences

| Antibody | Sequence | SEQ ID NO: |
|---|---|---|
| ALP-Tan- | EVQLVESGGGLVQAGDSLTLSCAASGRTFSSYAMGWFRQAPGKEREFVARITWTGRS | 1 |
| 3p-100 | TSYADSVKGRFTISRDNAKNRVYLRMNSLKPEDTAVYYCAADLEGAMVSRRREIEYG | |
| | HWGQGTQVTVSS | |
| ALP-Tan- | EVDLVESGGGLVQAGGSLRLSCAASGGSTFAMAWLRQAPGKEREFVAAVGRSPRSPG | 2 |
| 3p-101 | ITYYADSVKGRFTISRDNAKNTVYLQMNSLKPEDTAVYYCAAGGILGPRAHYDYWGQ | |
| | GTQVTVSS | |
| ALP-Tan- | QVQLVESGGGLVQAGGSLRLSCAASGRTFSRYAMGWFRQAPGKEREFVAAISWSGGT | 3 |
| 3p-102 | TNYADSVKGRFTISRDNAKITVYLQMNSLKPEDTAFYYCAAGKRLTLRSSGYKYWGQ | |
| | GTQVTVSS | |
| ALP-Tan- | AVQLVESGGGLVQAGGSLRLSCAASRGSTFAMAWIRQAPGKEREFVAAVGRSPRGPG | 4 |
| 3p-104 | ITYYADSVKGRFTISRDNANNTVYLQMNSLKPEDTAVYYCAAGGILGPRAQYDYWGQ | |
| | GTQVTVSS | |
| ALP-Tan- | QVQLVESGGGLVQAGSSLRLSCAASRGSTFAMAWIRQAPGKEREFVAAVGRSPRGPG | 5 |
| 3p-106 | ITYYADSVKGRFTISRDNANNTVYLQMNSLKPEDTAVYYCAAGGILGPRAQYDYWGQ | |
| | GTQVTVSS | |
| ALP-Tan- | QVQLVESGGGLVQPGGSLRLSCAASGSIFSSGTNFSDSKIDWYRQAPGKQRDWIAGI | 6 |
| 3p-112 | FSTGSTIYEDSVKGRFAISRDNAKNMGYLQMNSLKPEDTAVYYCRVIGRGILAWGQG | |
| | TQVTVSS | |
| ALP-Tan- | QVQLVESGGGLVQAGGSLRLSCAASRGSTFAMAWIRQAPRKEREFVPAVGRSPLGPV | 7 |
| 3p-118 | ITYYADSVKGRFTISRDNANNTVYLQMNSLKPEDTAVDYCASCGILGPRAHYDYWGQ | |
| | GTHVTVSS | |
| ALP-Tan- | EVQLVESGGGLVQAGDSLTLSCAASGRTFSSYAMGWFRQAPGKEREFVARITWSGRS | 8 |
| 3p-124 | TSYADSVKGRFTISRDNAKNRVYLRMNSLKPEDTAVYYCAADLEGAMVSRRREIEYG | |
| | QWGQGTQVTVSS | |
| ALP-Tan- | QVQLVESGGGLVQAGGSLRLSCAASGGSTFAMAWIRQAPGKEREFVAAVGRSPRSPG | 9 |
| 3p-127 | ITYYADSVKGRFTISRDNANNTVYLQMNSLKPEDTAVYYCAAGGILGPRAQYDYWGQ | |
| | GTQVTVSS | |
| ALP-Tan- | QVQLVESGGGLVQAGSSLRLSCAASRGSTFAMAWIRQAPGKEREFVAAVGRSPRGPG | 10 |
| 3p-89 | ITYYADSVKGRFTISRDNANNTVYLQMNSLKPEDTAVYYCAAGGILGPRAQYDYWGQ | |
| | GTQVTVSS | |
| ALP-Tan- | QVQLVESVGGLVQPGDSLRLSCLASGRTFTFRHYVMGWFRQAPGKEREFVAAISWSG | 11 |
| 3p-93 | SGSYYADSVKGRFTISRDNSKNMVFLQMNGLKPEDTAVYYCAADMTTRMSQASREYD | |
| | YWGQGTQVTVSS | |
| ALP-Tan- | EVQLVESGGGLVQAGGSLRLSCAASGGSTFAMAWLRQAPGKEREFVAAVGRSPRSPG | 12 |
| 3p-95 | ITYYADSVKGRFTISRDNAKNTVWLQMNSLKPEDTAVYYCAAGGILGPRAEYDYWGQ | |
| | GTRVTVSS | |
| ALP-Tan- | QVHLVESGGGLVQAGDSLTLSCAASGRTFSSYAMGWFRQAPGKEREFVARITWSGRS | 13 |
| 3p-99 | TSYADSVKGRFTISRDNAKNRVYLRMNSLKPEDTAVYYCAADLEGAMVSRRREIEYG | |
| | HWGQGTQVTVSS | |
| ASP-1P-1 | QVQLVESGGELVQAGGSLRLSCAASGRTFSSYAMGWFRQGPGKEREFVAAISASGGR | 14 |
| | TYYADSVKGRFTISRDNAKNTVYLQMNSLKPEDTAVYYCAVAGPRIRIATITLSREY | |
| | DYWGQGTQVTVSS | |
| ASP-3P-10 | AVQLVESGGGLVQAGGSLRLSCVASEIAFSVFDMGWYRQAPGKQRELAASIGHDGRI | 15 |
| | NYADSVKGRFTISRDNAKNTVHLQMNTLKSEDTAVYYCNARNSFRDLWGQGTQVTVS | |
| | S | |
| ASP-3P-13 | AVQLVESGGGLVQPGGSLRLSCAASGRSFSGYAMGWFRQAPGKEREFVSAISGSGRN | 16 |
| | TYYADSVKGRFTISRDNAKNTMYLQMNSLKPEDTAVYYCAVAGPAITIATMTLRGKY | |
| | DYWGQGTQVTVSS | |
| ASP-1P-2 | QVHLVESGGGLVQAGDSLRLSCAASGRTFSSRAMGWFRQAPGKEREFVAAISASGSR | 17 |
| | TYYADSVKGRFTISRDNAKNTVYLQMNSLKPEDTAVYYCAAAGPRITIATMTLSREY | |
| | DYWGQGTQVTVSS | |
| ASP-1P-3 | EVQLVESGGGLVQPGGSLRLSCAASGRTFSSYALGWFRQAPGKEREFVAAISASGLR | 18 |
| | TYYADSVKGRFTISRDNAKNTVYLQMNSLKPEDTAVYYCAEAGPRIRIATMTLSREY | |
| | DYWGQGTLVTVSS | |
| ASP-2P-4 | QVQLVESGGGLVQAGGSLRLSCAASGRTFSSYAMGWFRQAPGKEREFATAISASGRS | 19 |
| | TYYADSVKGRFTISRDNAKNTVYLQMNSLKPEDTAVYYCAQGGPSITIRTMGSSSKY | |
| | DYWGRGTQVTVSS | |
| ASP-2P-8 | QVHLVESGGGLVQAGGSLRLSCAASGRTFSSYAMGWFRQAPGKEREFVAAVSASGGR | 20 |
| | SYYVDSVKGRFTISRDNAKNTVYLQMNSLKPEDTAVYYCAAAGRSITIATMTERYKY | |
| | DYWGQGTQVTVSS | |
| ASP-2P-14 | EVQLVESGGGLVQAGGSLRLSCAASGRSFSGYAMGWFRQAPGKERDFVAAISGTGGS | 21 |
| | TYYVDSVKGRFTISRDNAKNTMYLQMNSLKPEDTAVYYCAVAGPAITIATMTLRGKY | |
| | DYWGQGTQVTVSS | |
| ASP-2P-15 | AVQLVESGGSLRLSCAASGRTFSSYAMGWFRQAPGKEREFVAAISGSGARTYYADSV | 22 |
| | KGRFTISRANTKNTVYLQMNSLKPEDTAVYYCAADATRIASVDVPKSWGYWGQGTQV | |
| | TVSS | |
| ASP-2P-17 | QLHFVESGGGLVQAGGSLRLACAASGRTFSGYARTWFRQAPGKEREFVAAISGSGAS | 23 |
| | AYYADSVKGRFTISRDNAKNTVYLQMNSLKPEDTAVYYCAADQSIRIATMRTHAAYG | |
| | YWGQGTQVTVSS | |
| ASP-2P-18 | QVQLVESVGGLVQAGGSLSLSCAASGGSTFAMAWLRQAPGKEREFVAAVGRSPRGPG | 24 |
| | ITNYADSVKGRFTISRDNAKNTVYLQMNSLKPEDTAVYYCAAGGILGPRAQYDYWGQ | |
| | GTRVTVSS | |
| ASP-3P-26 | EVHLVESGGGLVQPGGSLRLSCAHSGSIRSINVMNWYRQVPGKQRELVATITAGGST | 25 |
| | NYADSVKGRFTISRDNALNTAALQMNSLRPEDTAVYYCHADKILTYNGVIYRAEYDV | |
| | WGQGTQVTVSS | |
| ASP-3P-27 | QVQLVESGGGLVQAGGSLRLSCAASGRTFSGYAMGWFRQAPGKEREFVAAISGSGGR | 26 |
| | TYYVDSAKGRFTISRDNAKNTMYLQMNSLKPEDTAVYYCAVAGPAITIATMTLRGKY | |
| | DYWGQGTQVTVSS | |
| ASP-3P-29 | QVQLVESGGGLVQPGGSLRLSCAASESIRSINVMNWYRQAPGKQRELVATITSGGTT | 27 |
| | TYADSVKGRFTSSRDNAKNTVALQMNSLRPEDTAVYYCHADKVLYYNGVIYGAEYDV | |
| | WGQGTQVTVSS | |
| ASP-3P-30 | QLQLVESGGGLVQPGGSLRLSCAPSGSIRSINVMNWYRQAPGKQRDLVATITSGGST | 28 |
| | NYADSVKGRFIISRDNAKNTVALQMNSLRPEDTAVYYCHADKVLYYNGVLYGAEYDV | |
| | WGQGTQVTVSS | |
| ASP-3P-35 | EVHLVESGGGLVQAGGSLRLSCEVSGSIFSGTHFSFNTMGWYRQAPGKQRELVALGR | 29 |
| | GSRGINYADSVKGRFTFSSDNAKNTIFLQMNNLEPEDTGNYTCYVRRPSGSYAGQYY | |
| | PDSSEYWGQGTQVTVSS | |
| ASP-3P-36 | QPQVVESGGGLVQAGGSLRLSCVASGSNFAFEYIAWYRQAPGKEREVVALISPQSIT | 30 |
| | TYADSVKGRFTISRDNAKSTVYLQMNSLKPEDTAVYYCHDREYWGQGTQVTVSS | |
| ASP-3P-38 | QLHFVESGGGLVQPGGSLRLSCAASGSIRSINVMNYYRQAPGKQRELVATISSVGST | 31 |
| | NYADSVKGRFTISRDNAQNTVALQMNSLRPEDTAVYYCHADKVLYYNGVMYGVESDV | |
| | WGQGTQVTVSS | |
| ASP-3P-40 | QVQLVESGGGEVQPGGSLRLSCAASGPTFSRYIMGWFRQAPGKEREFVAAISRIGGI | 32 |
| | TYYTDSVKGRFTISRDNAKNTVYLQMNSLEPEDTASYYCAAKSSSSSSKYTARGADA | |
| | YDYWGQGTQVTVSS | |
| ASP-3P-43 | AVQLVESGGGLVQPGGSLRLSCAASRSVFSVLVMGWYRQAPGQQRELVATISNEGYS | 33 |
| | NYADSVKGRFAISRDNAKKTVYLQMNSLKPEDTAVYYCNAAWGNGRYTYWGQGTQVT | |
| | VSS | |
| ASP-3P-44 | EVHLVESGGGLVQPGGSLRLSCAHSGSIRSINVMNWYRQVPGKQRELVATITAGGST | 34 |
| | NYADSVKGRFTISRDNAKNSAALQMNSLRPEDTAVYYCHADKVLSYNGVIYRAEYDV | |
| | WGQGTQVTVSS | |
| ASP-3P-45 | ELQLVESGGGLVQAGGSLRLSCAVSESIFKFPRMGWYRQGPGDQRDLVALSRSSGST | 35 |
| | EYADFAKGRFTISRDNSKNTVYLQMNSLKPEDSGTYYCYVRRPSGSENGRWYTDPSD | |
| | DWGQGTRVTVSS | |
| ASP-3P-46 | QVQLVESGGGLVQPGGSLRLSCAASARSINGMEWYRQAPGERRELVAGITAGGSAYY | 36 |
| | TDTVKGRFTISRDNAENTGYLQMNSLSPDDTAVYYCRRQYGPNWYWGQGTQVTVSS | |

### Example 2. Binding and Blocking Activity of Alpaca Monoclonal Antibodies Against Human PD-L1

The binding and blocking property of some of the antibodies were characterized by Gator. Anti-his probe was first loaded onto the chip and followed by human PD-L1-his to capture the antigen. Then, the antibodies were injected to record the binding curve. Finally, human PD1/hFc was injected to determine whether the antibodies could block the interaction between PD-1 and PD-L1. All of ALP-Tan-3p-112, ALP-Tan-3p-93 and ASP-30-46 effectively blocked the interaction between PD-1 and PD-L1 (Table 2). The affinity was further confirmed by Biacore T200.

TABLE 2
Affinities

| | Kon (1/Ms) | Koff (1/s) | KD (M) |
|---|---|---|---|
| ALP-Tan-3p-112 | 4.9 × 10⁵ | 0.000235 | 4.79 × 10⁻¹⁰ |
| ALP-Tan-3p-93 | 1.54 × 10⁵ | 7.8 × 10⁻⁵ | 4.59 × 10⁻¹⁰ |
| ASP-3p-46 | 7.1 × 10⁵ | 0.00128 | 1.81 × 10⁻⁹ |

### Example 3. Humanization of Anti-PD-L1 Alpaca Monoclonal Antibodies

The mAb ALP-Tan-3p-93 and ALP-Tan-3p-112 variable region genes were employed to create a humanized mAb. In the first step of this process, the amino acid sequences of the ALP-Tan-3p-93 and ALP-Tan-3p-112 were compared against the available database of human Ig gene sequences to find the overall best-matching human germline Ig gene sequences. For ALP-Tan-3p-93, the closest human match was IGHV3-23*04 gene. Humanized variable domain sequences were then designed where the CDR1, 2 and 3 of the ALP-Tan-3p-93 were grafted onto framework sequences of the IGHV3-23*04 gene. For ALP-Tan-3p-112, the closest human match was IGHV3-48*03 gene. Humanized variable domain sequences were then designed where the CDR1, 2 and 3 of the ALP-Tan-3p-112 were grafted onto framework sequences of the IGHV3-48*03 gene. Meanwhile, one residue mutation (N34Q, Kabat numbering) was introduced into CDR1 to reduce the risk of posttranslational modification. A 3D model was then generated to determine if there were any framework positions where replacing the alpaca amino acid to the human amino acid could affect binding and/or CDR conformation.

TABLE 3
Humanized antibodies and back mutations

| Chain | Sequence | SEQ ID NO: |
|---|---|---|
| 93_VH-1 | EVQLVESGGGLVQPGGSLRLSCAASGFTFTFRHYVMGWVRQAPGKEREWVSA | 37 |
| | ISWSGSGSYYADSVKGRFTISRDNSKNTLYLQMNSLRAEDTAVYYCAKDMTT | |
| | RMSQASREYDYWGQGTLVTVSS | |
| 93_VH-2 | EVQLVESGGGLVQPGGSLRLSCAASGFTFTFRHYVMGWVRQAPGKEREWVSA | 38 |
| | ISWSGSGSYYADSVKGRFTISRDNSKNTVYLQMNSLRAEDTAVYYCAADMTT | |
| | RMSQASREYDYWGQGTLVTVSS | |
| 93_VH-3 | EVQLVESGGGLVQPGGSLRLSCAASGFTFTFRHYVMGWFRQAPGKGLEFVAA | 39 |
| | ISWSGSGSYYADSVKGRFTISRDNSKNTLYLQMNSLRAEDTAVYYCAKDMTT | |
| | RMSQASREYDYWGQGTLVTVSS | |
| 93_VH-4 | EVQLVESGGGLVQPGGSLRLSCAASGFTFTFRHYVMGWFRQAPGKGLEFVAA | 40 |
| | ISWSGSGSYYADSVKGRFTISRDNSKNTVYLQMNSLRAEDTAVYYCAADMTT | |
| | RMSQASREYDYWGQGTLVTVSS | |
| 93_VH-5 | EVQLVESGGGLVQPGGSLRLSCAASGFTFTFRHYVMGWFRQAPGKEREFVAA | 41 |
| | ISWSGSGSYYADSVKGRFTISRDNSKNTLYLQMNSLRAEDTAVYYCAKDMTT | |
| | RMSQASREYDYWGQGTLVTVSS | |
| 93_VH-6 | EVQLVESGGGLVQPGGSLRLSCAASGFTFTFRHYVMGWFRQAPGKEREFVAA | 42 |
| | ISWSGSGSYYADSVKGRFTISRDNSKNTVYLQMNSLRAEDTAVYYCAADMTT | |
| | RMSQASREYDYWGQGTLVTVSS | |
| 93_VH-7 | EVQLVESGGGLVQPGGSLRLSCAASGFTFTFRHYVMGWFRQAPGKGLEFVAA | 43 |
| | ISWSGSGSYYADSVKGRFTISRDNSKNTLYLQMNSLRAEDTAVYYCAADMTT | |
| | RMSQASREYDYWGQGTLVTVSS | |
| 93_VH-8 | EVQLVESGGGLVQPGGSLRLSCAASGRTFTFRHYVMGWFRQAPGKGLEFVAA | 44 |
| | ISWSGSGSYYADSVKGRFTISRDNSKNTVYLQMNSLRAEDTAVYYCAADMTT | |
| | RMSQASREYDYWGQGTLVTVSS | |
| 93_VH-9 | EVQLVESGGGLVQPGGSLRLSCAASGRTFTFRHYVMGWFRQAPGKGLEFVAA | 45 |
| | ISWSGSGSYYADSVKGRFTISRDNSKNTLYLQMNSLRAEDTAVYYCAADMTT | |
| | RMSQASREYDYWGQGTLVTVSS | |
| 112-VHH1- | EVQLVESGGGLVQPGGSLRLSCAASGSIFSSGTQFSDSKIDWVRQAPGKGLE | 46 |
| PTM | WVSGIFSTGSTIYEDSVKGRFTISRDNAKNSLYLQMNSLRAEDTAVYYCARI | |
| | GRGILAWGQGTLVTVSS | |
| 112-VHH2- | EVQLVESGGGLVQPGGSLRLSCAASGSIFSSGTQFSDSKIDWYRQAPGKQRE | 47 |
| PTM | WVSGIFSTGSTIYEDSVKGRFTISRDNAKNSLYLQMNSLRAEDTAVYYCARI | |
| | GRGILAWGQGTLVTVSS | |
| 112-VHH3- | EVQLVESGGGLVQPGGSLRLSCAASGSIFSSGTQFSDSKIDWYRQAPGKQRE | 48 |
| PTM | WVSGIFSTGSTIYEDSVKGRFTISRDNAKNSLYLQMNSLRAEDTAVYYCRVI | |
| | GRGILAWGQGTLVTVSS | |
| 112-VHH4- | EVQLVESGGGLVQPGGSLRLSCAASGSIFSSGTQFSDSKIDWYRQAPGKQRE | 49 |
| PTM | WVAGIFSTGSTIYEDSVKGRFTISRDNAKNSLYLQMNSLRAEDTAVYYCRVI | |
| | GRGILAWGQGTLVTVSS | |
| 112-VHH5- | EVQLVESGGGLVQPGGSLRLSCAASGSIFSSGTQFSDSKIDWYRQAPGKQRD | 50 |
| PTM | WVAGIFSTGSTIYEDSVKGRFAISRDNAKNSLYLQMNSLRAEDTAVYYCRVI | |
| | GRGILAWGQGTLVTVSS | |
| 112-VHH6- | EVQLVESGGGLVQPGGSLRLSCAASGSIFSSGTQFSDSKIDWYRQAPGKQRE | 51 |
| PTM | WVAGIFSTGSTIYEDSVKGRFTISRDNAKNMLYLQMNSLRAEDTAVYYCRVI | |
| | GRGILAWGQGTLVTVSS | |
| 112-VHH7- | EVQLVESGGGLVQPGGSLRLSCAASGSIFSSGTQFSDSKIDWYRQAPGKQRE | 52 |
| PTM | WIAGIFSTGSTIYEDSVKGRFAISRDNAKNMLYLQMNSLRAEDTAVYYCRVI | |
| | GRGILAWGQGTLVTVSS | |
| 112-VH47 | EVQLVESGGGLVQPGGSLRLSCAASGFTFSSGTQFSDSKIDWYRQAPGKGLV | 53 |
| | WVAGIFSTGSTIYEDSVKGRFTISRDNAKNTGYLQMNSLRAEDTAVYYCRVI | |
| | GRGILAWGQGTLVTVSS | |

TABLE 3A
CDR sequences

| Antibody series | CDRs | SEQ ID NO: |
|---|---|---|
| 93 | FRHYVMG | 54 |
| | AISWSGSGSYYADSVKG | 55 |
| | DMTTRMSQASREYDY | 56 |
| 112 | SGTQFSDSKID | 57 |
| | GIFSTGSTIYEDSVKG | 58 |
| | IGRGILA | 59 |

### Example 4. Full Kinetics of Humanized Anti-PD-L1 Monoclonal Antibodies

To explore the binding kinetics of the humanized antibody, this example further performed the full kinetic affinity testing by monitoring association and dissociation of various dose of antigen (100 nM, 50 nM, 25 nM, 12.5 nM, 6.15 nM, 3.125 nM, 1.5625 nM) against different monoclonal antibodies by Biacore. As shown Table 4, 112-VHH5-PTM affinity was comparable with ALP-Tan-3p-112 chimeric antibody. 93VH-4, 93VH-6 and 93VH-8 affinity were comparable with ALP-Tan-3p-93 chimeric antibody.

TABLE 4
Affinities

| | Kon (1/Ms) | Koff (1/s) | KD (M) |
|---|---|---|---|
| ALP-Tan-3p-112 (chimeric) | 2.14 × 10⁶ | 5.66 × 10⁻⁴ | 2.64 × 10⁻¹⁰ |
| 112-VHH3-PTM | 2.07 × 10⁶ | 4.98 × 10⁻³ | 2.40 × 10⁻⁹ |
| 112-VHH5-PTM | 1.62 × 10⁶ | 1.49 × 10⁻³ | 9.20 × 10⁻¹⁰ |
| 112-VHH7-PTM | 1.69 × 10⁶ | 3.93 × 10⁻³ | 2.32 × 10⁻⁹ |
| ALP-Tan-3p-93 (chimeric) | 1.92 × 10⁵ | 1.07 × 10⁻⁴ | 5.59 × 10⁻¹⁰ |
| 93-VH-2 | 6.15 × 10⁴ | 1.62 × 10⁻³ | 2.63 × 10⁻⁸ |
| 93-VH-3 | 4.86 × 10⁵ | 1.15 × 10⁻³ | 2.36 × 10⁻⁹ |
| 93-VH-4 | 8.05 × 10⁴ | 1.47 × 10⁻⁴ | 1.82 × 10⁻⁹ |
| 93-VH-5 | 4.52 × 10⁵ | 1.25 × 10⁻³ | 2.77 × 10⁻⁹ |
| 93-VH-6 | 9.59 × 10⁴ | 1.97 × 10⁻⁴ | 2.06 × 10⁻⁹ |
| 93-VH-7 | 8.76 × 10⁴ | 3.39 × 10⁻⁴ | 3.87 × 10⁻⁹ |
| 93-VH-8 | 1.33 × 10⁵ | 2.39 × 10⁻⁴ | 1.80 × 10⁻⁹ |
| 93-VH-9 | 1.40 × 10⁵ | 3.10 × 10⁻⁴ | 2.20 × 10⁻⁹ |

### Example 5. Binding Properties of the Humanized Anti-PD-L1 Antibody

Binding properties of the humanized anti-PD-L1 antibody of the present application were first evaluated by ELISA assay. Briefly, 100 μl anti-PD-L1 antibody 93-VH6 or 112-VH47 at different concentrations was incubated in each well of 96 well plate pre-coated with human His-PD-L1, and then goat anti-human IgG Fc HRP was added and analyzed by coloring reaction of HRP with its substrate. The exemplary anti-PD-L1 antibody 93-VH6 and 112-VH47 both displayed specific binding with human PD-L1 in a dose dependent manner.

Binding capability of the anti-PD-L1 antibody of the present application was further evaluated by using Raji cells overexpressing human PD-L1. Briefly, 50 μl Raji cells overexpressing human PD-L1 were seeded into 96 well plate at a concentration of 2*10⁵ cells/well. 50 μl anti-PD-L1 antibody 93-VH6 or 112-VH47 at different concentrations was added into each well and incubated with the cells on ice for 1 hour. Then the cells were washed twice by FACS buffer and supplemented with 100 μl PE-anti-hu IgG, followed by incubation on ice for 1 hour. After incubation, the cells in each well were collected and resuspended with 65 μl FACS buffer for analysis by flow cytometry. The exemplary anti-PD-L1 antibody 93-VH6 and 112-VH47 both displayed specific binding with Raji cells overexpressing human PD-L1 in a dose dependent manner.

### Example 6. T Cell Activation Bioassay (NFAT)

To test the ability of the anti-PD-L1 antibodies to stimulate T cell response, hPD-1-expressed Jurkat cells were used. Jurkat is a human T cell leukemia cell line that can activate NFAT-mediated luciferase expression upon TCR stimulation. In this assay, Jurkat cells transfected with human PD-1 gene by lentivirus were used as the responder cells. The Raji-PD-L1 cells was used as the antigen presenting cells (APC). Staphylococcal Enterotoxin E (SEE) is used to stimulate TCR signal. In this system, ectopically expressed huPD-L1 can suppress SEE stimulated NFAT-luciferase activity in Jurkat cells, while anti-PD-L1 antibodies can reverse NFAT-luciferase activity. In short, APCs (2.5×10⁴) were co-cultured with PD-1 expressing Jurkat T cells (1×10⁵) in the presence of SEE stimulation. Anti-PD-L1 antibodies were added at the beginning of the culture. Six hours later, the resulting cells were evaluated for its luciferase activity.

All anti-PD-L1 antibodies tested blocked PD-1/PD-L1 interaction thus enhanced NFAT-mediated luciferase activity.

### Example 7. Affinity Maturation of 93-VH6 and 112-VH47

Two anti-PD-L1 single domain antibodies (sdAb) 93-VH6 and 112-VH47 were subjected to affinity maturation. One, two or three residues in each CDRs were selected for mutation (see mutated CDRs in Table 5A) A total of 30 candidate antibodies were designed and made using synthesized cDNA. Their sequences, as well as the mutated CDRs are listed in Table 5B-C below.

TABLE 5A
Affinity Maturated CDRs

| | | SEQ | | SEQ |
|---|---|---|---|---|
| Anti- | | ID | | ID |
| body | CDRs | NO: | Mutated CDR | NO: |
| 93- | FRHYVMG | 54 | FYHYVMG | 95 |
| VH6 | AISWSGSGSYYADSVKG | 55 | DISWSGSGSYYADSVKG | 96 |
| | DMTTRMSQASREYDY | 56 | DMTTEMSQASREYDY | 97 |
| | | | DMTTRMSQDSREYDY | 98 |
| | | | DMTTRMSQASDEYDY | 99 |
| | | | DMTTEMSQDSDEYDY | 100 |
| | | | DMTTEMSQDSREYDY | 101 |
| | | | DMTTEMSQASDEYDY | 102 |
| | | | DMTTRMSQDSDEYDY | 103 |
| 112- | SGTQFSDSKID | 57 | SGTQFSDSKAD | 90 |
| VH47 | GIFSTGSTIYEDSVKG | 58 | GIFQTGSTIYEDSVKG | 91 |
| | IGRGILA | 59 | IGIGILA | 92 |
| | | | IGRGTLA | 93 |
| | | | IGIGTLA | 94 |

TABLE 5B
Affinity Maturation

| Antibody | Sequence (mutations underlined) | SEQ ID NO: |
|---|---|---|
| R21560101 | EVQLVESGGGLVQPGGSLRLSCAASGFTFSSGTQFSDSKADWYRQAPGKGLV | 60 |
| | WVAGIFSTGSTIYEDSVKGRFTISRDNAKNTGYLQMNSLRAEDTAVYYCRVI | |
| | GRGILAWGQGTLVTVSS | |
| R21560102 | EVQLVESGGGLVQPGGSLRLSCAASGFTFSSGTQFSDSKIDWYRQAPGKGLV | 61 |
| | WVAGIFQTGSTIYEDSVKGRFTISRDNAKNTGYLQMNSLRAEDTAVYYCRVI | |
| | GRGILAWGQGTLVTVSS | |
| R21560103 | EVQLVESGGGLVQPGGSLRLSCAASGFTFSSGTQFSDSKIDWYRQAPGKGLV | 62 |
| | WVAGIFSTGSTIYEDSVKGRFTISRDNAKNTGYLQMNSLRAEDTAVYYCRVI | |
| | GIGILAWGQGTLVTVSS | |
| R21560104 | EVQLVESGGGLVQPGGSLRLSCAASGFTFSSGTQFSDSKIDWYRQAPGKGLV | 63 |
| | WVAGIFSTGSTIYEDSVKGRFTISRDNAKNTGYLQMNSLRAEDTAVYYCRVI | |
| | GRGTLAWGQGTLVTVSS | |
| R21560105 | EVQLVESGGGLVQPGGSLRLSCAASGFTFSSGTQFSDSKADWYRQAPGKGLV | 64 |
| | WVAGIFSTGSTIYEDSVKGRFTISRDNAKNTGYLQMNSLRAEDTAVYYCRVI | |
| | GIGILAWGQGTLVTVSS | |
| R21560106 | EVQLVESGGGLVQPGGSLRLSCAASGFTFSSGTQFSDSKADWYRQAPGKGLV | 65 |
| | WVAGIFSTGSTIYEDSVKGRFTISRDNAKNTGYLQMNSLRAEDTAVYYCRVI | |
| | GRGTLAWGQGTLVTVSS | |
| R21560107 | EVQLVESGGGLVQPGGSLRLSCAASGFTFSSGTQFSDSKIDWYRQAPGKGLV | 66 |
| | WVAGIFQTGSTIYEDSVKGRFTISRDNAKNTGYLQMNSLRAEDTAVYYCRVI | |
| | GIGILAWGQGTLVTVSS | |
| R21560108 | EVQLVESGGGLVQPGGSLRLSCAASGFTFSSGTQFSDSKIDWYRQAPGKGLV | 67 |
| | WVAGIFQTGSTIYEDSVKGRFTISRDNAKNTGYLQMNSLRAEDTAVYYCRVI | |
| | GRGTLAWGQGTLVTVSS | |
| R21560109 | EVQLVESGGGLVQPGGSLRLSCAASGFTFSSGTQFSDSKIDWYRQAPGKGLV | 68 |
| | WVAGIFSTGSTIYEDSVKGRFTISRDNAKNTGYLQMNSLRAEDTAVYYCRVI | |
| | GIGTLAWGQGTLVTVSS | |
| R21560110 | EVQLVESGGGLVQPGGSLRLSCAASGFTFSSGTQFSDSKADWYRQAPGKGLV | 69 |
| | WVAGIFQTGSTIYEDSVKGRFTISRDNAKNTGYLQMNSLRAEDTAVYYCRVI | |
| | GIGTLAWGQGTLVTVSS | |
| R21560111 | EVQLVESGGGLVQPGGSLRLSCAASGFTFSSGTQFSDSKADWYRQAPGKGLV | 70 |
| | WVAGIFQTGSTIYEDSVKGRFTISRDNAKNTGYLQMNSLRAEDTAVYYCRVI | |
| | GIGILAWGQGTLVTVSS | |
| R21560112 | EVQLVESGGGLVQPGGSLRLSCAASGFTFSSGTQFSDSKADWYRQAPGKGLV | 71 |
| | WVAGIFQTGSTIYEDSVKGRFTISRDNAKNTGYLQMNSLRAEDTAVYYCRVI | |
| | GRGTLAWGQGTLVTVSS | |
| R21448502 | EVQLVESGGGLVQPGGSLRLSCAASGFTFTFYHYVMGWFRQAPGKEREFVAA | 72 |
| | ISWSGSGSYYADSVKGRFTISRDNSKNTVYLQMNSLRAEDTAVYYCAADMTT | |
| | RMSQASREYDYWGQGTLVTVSS | |
| R21448503 | EVQLVESGGGLVQPGGSLRLSCAASGFTFTFRHYVMGWFRQAPGKEMEFVAD | 73 |
| | ISWSGSGSYYADSVKGRFTISRDNSKNTVYLQMNSLRAEDTAVYYCAADMTT | |
| | RMSQASREYDYWGQGTLVTVSS | |
| R21448504 | EVQLVESGGGLVQPGGSLRLSCAASGFTFTFRHYVMGWFRQAPGKEREFVAA | 74 |
| | ISWSGSGSYYADSVKGRFTISRDNSKNTVYLQMNSLRAEDTAVYYCAADMTT | |
| | EMSQASREYDYWGQGTLVTVSS | |
| R21448505 | EVQLVESGGGLVQPGGSLRLSCAASGFTFTFRHYVMGWFRQAPGKEREFVAA | 75 |
| | ISWSGSGSYYADSVKGRFTISRDNSKNTVYLQMNSLRAEDTAVYYCAADMTT | |
| | RMSQDSREYDYWGQGTLVTVSS | |
| R21448506 | EVQLVESGGGLVQPGGSLRLSCAASGFTFTFRHYVMGWFRQAPGKEREFVAA | 76 |
| | ISWSGSGSYYADSVKGRFTISRDNSKNTVYLQMNSLRAEDTAVYYCAADMTT | |
| | RMSQASDEYDYWGQGTLVTVSS | |
| R21448507 | EVQLVESGGGLVQPGGSLRLSCAASGFTFTFYHYVMGWFRQAPGKEREFVAA | 77 |
| | ISWSGSGSYYADSVKGRFTISRDNSKNTVYLQMNSLRAEDTAVYYCAADMTT | |
| | EMSQASREYDYWGQGTLVTVSS | |
| R21448508 | EVQLVESGGGLVQPGGSLRLSCAASGFTFTFYHYVMGWFRQAPGKEREFVAA | 78 |
| | ISWSGSGSYYADSVKGRFTISRDNSKNTVYLQMNSLRAEDTAVYYCAADMTT | |
| | RMSQDSREYDYWGQGTLVTVSS | |
| R21448509 | EVQLVESGGGLVQPGGSLRLSCAASGFTFTFYHYVMGWFRQAPGKEREFVAA | 79 |
| | ISWSGSGSYYADSVKGRFTISRDNSKNTVYLQMNSLRAEDTAVYYCAADMTT | |
| | RMSQASDEYDYWGQGTLVTVSS | |
| R21448510 | EVQLVESGGGLVQPGGSLRLSCAASGFTFTFRHYVMGWFRQAPGKEMEFVAD | 80 |
| | ISWSGSGSYYADSVKGRFTISRDNSKNTVYLQMNSLRAEDTAVYYCAADMTT | |
| | EMSQASREYDYWGQGTLVTVSS | |
| R21448511 | EVQLVESGGGLVQPGGSLRLSCAASGFTFTFRHYVMGWFRQAPGKEMEFVAD | 81 |
| | ISWSGSGSYYADSVKGRFTISRDNSKNTVYLQMNSLRAEDTAVYYCAADMTT | |
| | RMSQDSREYDYWGQGTLVTVSS | |
| R21448512 | EVQLVESGGGLVQPGGSLRLSCAASGFTFTFRHYVMGWFRQAPGKEMEFVAD | 82 |
| | ISWSGSGSYYADSVKGRFTISRDNSKNTVYLQMNSLRAEDTAVYYCAADMTT | |
| | RMSQASDEYDYWGQGTLVTVSS | |
| R21448513 | EVQLVESGGGLVQPGGSLRLSCAASGFTFTFYHYVMGWFRQAPGKEREFVAD | 83 |
| | ISWSGSGSYYADSVKGRFTISRDNSKNTVYLQMNSLRAEDTAVYYCAADMTT | |
| | EMSQASREYDYWGQGTLVTVSS | |
| R21448514 | EVQLVESGGGLVQPGGSLRLSCAASGFTFTFYHYVMGWFRQAPGKEREFVAD | 84 |
| | ISWSGSGSYYADSVKGRFTISRDNSKNTVYLQMNSLRAEDTAVYYCAADMTT | |
| | RMSQDSREYDYWGQGTLVTVSS | |
| R21448515 | EVQLVESGGGLVQPGGSLRLSCAASGFTFTFYHYVMGWFRQAPGKEREFVAD | 85 |
| | ISWSGSGSYYADSVKGRFTISRDNSKNTVYLQMNSLRAEDTAVYYCAADMTT | |
| | RMSQASDEYDYWGQGTLVTVSS | |
| R21448516 | EVQLVESGGGLVQPGGSLRLSCAASGFTFTFYHYVMGWFRQAPGKEREFVAD | 86 |
| | ISWSGSGSYYADSVKGRFTISRDNSKNTVYLQMNSLRAEDTAVYYCAADMTT | |
| | EMSQDSDEYDYWGQGTLVTVSS | |
| R21448517 | EVQLVESGGGLVQPGGSLRLSCAASGFTFTFRHYVMGWFRQAPGKEREFVAA | 87 |
| | ISWSGSGSYYADSVKGRFTISRDNSKNTVYLQMNSLRAEDTAVYYCAADMTT | |
| | EMSQDSREYDYWGQGTLVTVSS | |
| R21448518 | EVQLVESGGGLVQPGGSLRLSCAASGFTFTFRHYVMGWFRQAPGKEREFVAA | 88 |
| | ISWSGSGSYYADSVKGRFTISRDNSKNTVYLQMNSLRAEDTAVYYCAADMTT | |
| | EMSQASDEYDYWGQGTLVTVSS | |
| R21448519 | EVQLVESGGGLVQPGGSLRLSCAASGFTFTFRHYVMGWFRQAPGKEREFVAA | 89 |
| | ISWSGSGSYYADSVKGRFTISRDNSKNTVYLQMNSLRAEDTAVYYCAADMTT | |
| | RMSQDSDEYDYWGQGTLVTVSS | |

TABLE 5C
CDR sequences

| | | SEQ | | SEQ | | SEQ |
|---|---|---|---|---|---|---|
| | | ID | | ID | | ID |
| Antibody | CDR1 | NO: | CDR2 | NO: | CDR3 | NO: |
| R21560101 | SGTQFSDSKAD | 90 | GIFSTGSTIYEDSVKG | 58 | IGRGILA | 59 |
| R21560102 | SGTQFSDSKID | 57 | GIFQTGSTIYEDSVKG | 91 | IGRGILA | 59 |
| R21560103 | SGTQFSDSKID | 57 | GIFSTGSTIYEDSVKG | 58 | IGIGILA | 92 |
| R21560104 | SGTQFSDSKID | 57 | GIFSTGSTIYEDSVKG | 58 | IGRGTLA | 93 |
| R21560105 | SGTQFSDSKAD | 90 | GIFSTGSTIYEDSVKG | 58 | IGIGILA | 92 |
| R21560106 | SGTQFSDSKAD | 90 | GIFSTGSTIYEDSVKG | 58 | IGRGTLA | 93 |
| R21560107 | SGTQFSDSKID | 57 | GIFQTGSTIYEDSVKG | 91 | IGIGILA | 92 |
| R21560108 | SGTQFSDSKID | 57 | GIFQTGSTIYEDSVKG | 91 | IGRGTLA | 93 |
| R21560109 | SGTQFSDSKID | 57 | GIFSTGSTIYEDSVKG | 58 | IGIGTLA | 94 |
| R21560110 | SGTQFSDSKAD | 90 | GIFQTGSTIYEDSVKG | 91 | IGIGTLA | 94 |
| R21560111 | SGTQFSDSKAD | 90 | GIFQTGSTIYEDSVKG | 91 | IGIGILA | 92 |
| R21560112 | SGTQFSDSKAD | 90 | GIFQTGSTIYEDSVKG | 91 | IGRGTLA | 93 |
| R21448502 | FYHYVMG | 95 | AISWSGSGSYYADSVKG | 55 | DMTTRMSQASREYDY | 56 |
| R21448503 | FRHYVMG | 54 | DISWSGSGSYYADSVKG | 96 | DMTTRMSQASREYDY | 56 |
| R21448504 | FRHYVMG | 54 | AISWSGSGSYYADSVKG | 55 | DMTTEMSQASREYDY | 97 |
| R21448505 | FRHYVMG | 54 | AISWSGSGSYYADSVKG | 55 | DMTTRMSQDSREYDY | 98 |
| R21448506 | FRHYVMG | 54 | AISWSGSGSYYADSVKG | 55 | DMTTRMSQASDEYDY | 99 |
| R21448507 | FYHYVMG | 95 | AISWSGSGSYYADSVKG | 55 | DMTTEMSQASREYDY | 97 |
| R21448508 | FYHYVMG | 95 | AISWSGSGSYYADSVKG | 55 | DMTTRMSQDSREYDY | 98 |
| R21448509 | FYHYVMG | 95 | AISWSGSGSYYADSVKG | 55 | DMTTRMSQASDEYDY | 99 |
| R21448510 | FRHYVMG | 54 | DISWSGSGSYYADSVKG | 96 | DMTTEMSQASREYDY | 97 |
| R21448511 | FRHYVMG | 54 | DISWSGSGSYYADSVKG | 96 | DMTTRMSQDSREYDY | 98 |
| R21448512 | FRHYVMG | 54 | DISWSGSGSYYADSVKG | 96 | DMTTRMSQASDEYDY | 99 |
| R21448513 | FYHYVMG | 95 | DISWSGSGSYYADSVKG | 96 | DMTTEMSQASREYDY | 97 |
| R21448514 | FYHYVMG | 95 | DISWSGSGSYYADSVKG | 96 | DMTTRMSQDSREYDY | 98 |
| R21448515 | FYHYVMG | 95 | DISWSGSGSYYADSVKG | 96 | DMTTRMSQASDEYDY | 99 |
| R21448516 | FYHYVMG | 95 | DISWSGSGSYYADSVKG | 96 | DMTTEMSQDSDEYDY | 100 |
| R21448517 | FRHYVMG | 54 | AISWSGSGSYYADSVKG | 55 | DMTTEMSQDSREYDY | 101 |
| R21448518 | FRHYVMG | 54 | AISWSGSGSYYADSVKG | 55 | DMTTEMSQASDEYDY | 102 |
| R21448519 | FRHYVMG | 54 | AISWSGSGSYYADSVKG | 55 | DMTTRMSQDSDEYDY | 103 |

### Example 8. Testing of Affinity Matured Antibodies

The affinity maturated antibodies were subjected to various testing for their binding affinity to the target PD-L1 protein.

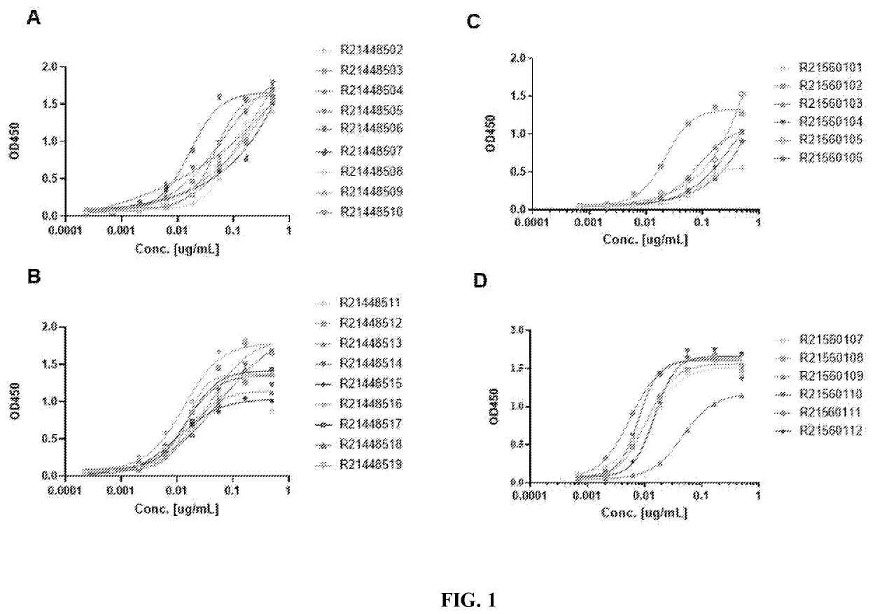

The ELISA testing results are shown in FIG. 1. Briefly, 100 μl anti-PD-L1 antibody at different concentrations was incubated in each well of 96 well plate pre-coated with human His-PD-L1, and then goat anti-human IgG Fc HRP was added and analyzed by coloring reaction of HRP with its substrate. The affinity matured anti-PD-L1 antibodies displayed specific binding with human PD-L1 in a dose dependent manner.

To explore the binding kinetics of the affinity matured antibody, the full kinetic affinity testing by monitoring association and dissociation of various dose of antigen (50 nM, 25 nM, 12.5 nM, 6.15 nM, 3.125 nM, 1.5625 nM) against different monoclonal antibodies by Biacore was further performed. The affinity maturated antibodies had similar or improved binding to human PD-L1 as compared to the parental antibodies.

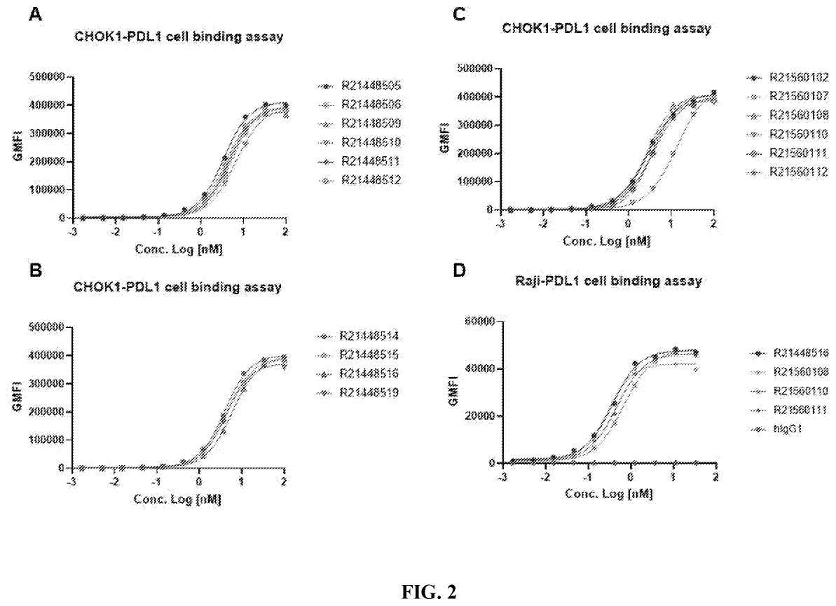

These antibodies' activities were also tested with respect to binding to PD-L1 expressed on CHO or Raji cells. As shown in FIG. 2, all of these antibodies showed good binding effect on cell surface PD-L1. The experiment was performed with similar method to Example 5.

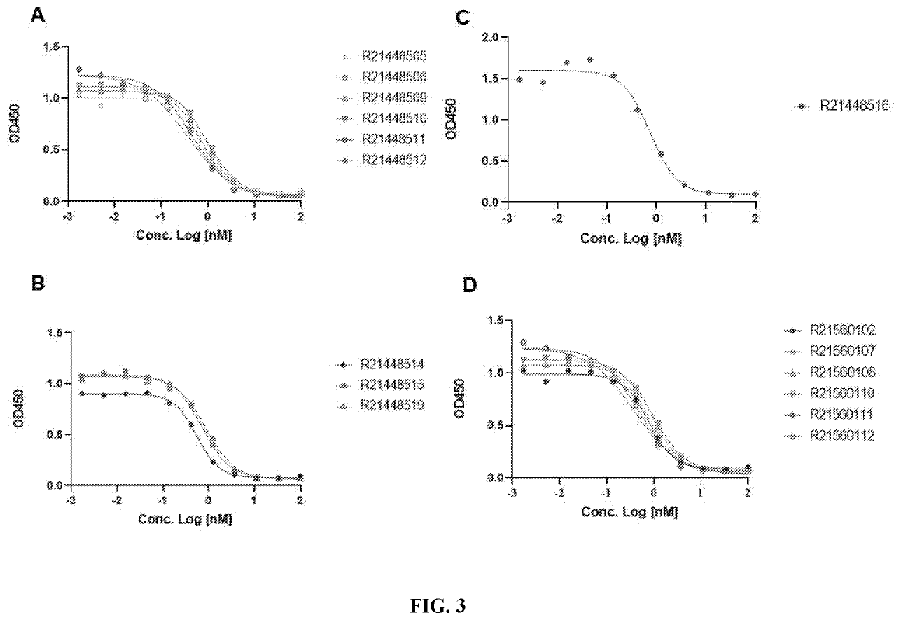

The antibodies' ability to block PD-L1 and PD-1 binding was tested with ELISA blocking assay. Briefly, 100 μl anti-PD-L1 antibody at different concentrations and Biotin-PD-1 were co-incubated in each well of 96 well plate pre-coated with human His-PD-L1, and then Streptavidin-HRP was added and analyzed by coloring reaction of HRP with its substrate. As shown in FIG. 3, all of these antibodies retained the high blocking activity.

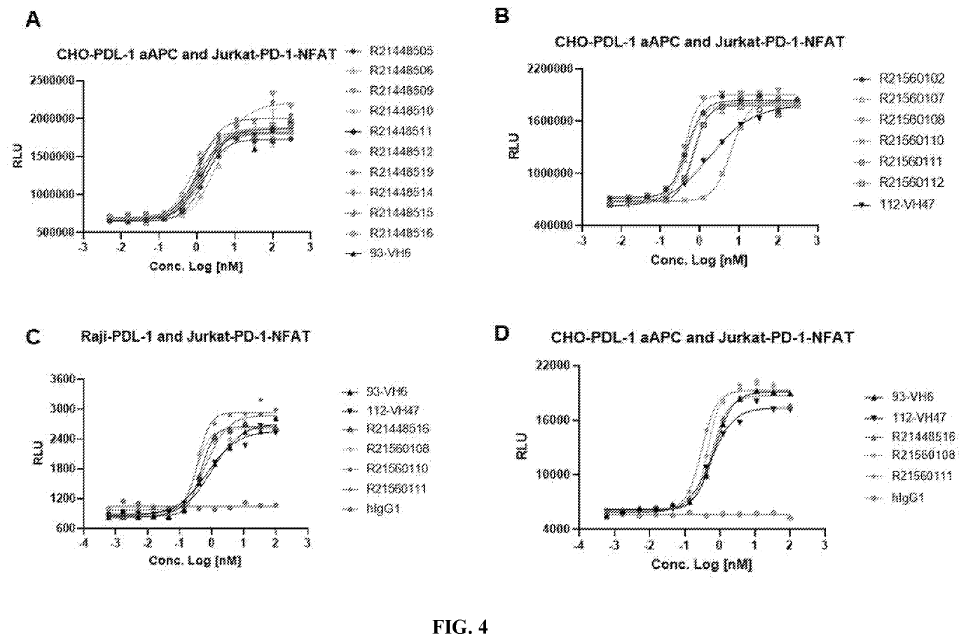

The biological function of these affinity matured antibodies was tested with the PD-1/PD-L1 Blockade bioassay. Briefly, CHO-K1 cells expressing human PD-L1 and an engineered cell surface protein was used as target cell, Jurkat T cells expressing human PD-1 and a luciferase reporter driven by an NFAT-response element (NFAT-RE) was used as effector cell. Anti-PD-L1 antibody at different concentrations were incubated with these two cells at 37° for 6 hours. Six hours later, the resulting cells were evaluated for its luciferase activity. As shown in FIG. 4A-C, all of the antibodies were biologically active. NFAT T cell activation assay as described in Example 6 was also performed, and similar results were generated as shown in FIG. 4D. All affinity matured anti-PD-L1 antibodies tested blocked PD-1/PD-L1 interaction thus enhanced NFAT-mediated luciferase activity. And the affinity matured antibodies, e.g., R21448516, R21560108, R21560111 showed improved effect over their parental molecule 93-VH6 and 112-VH47.

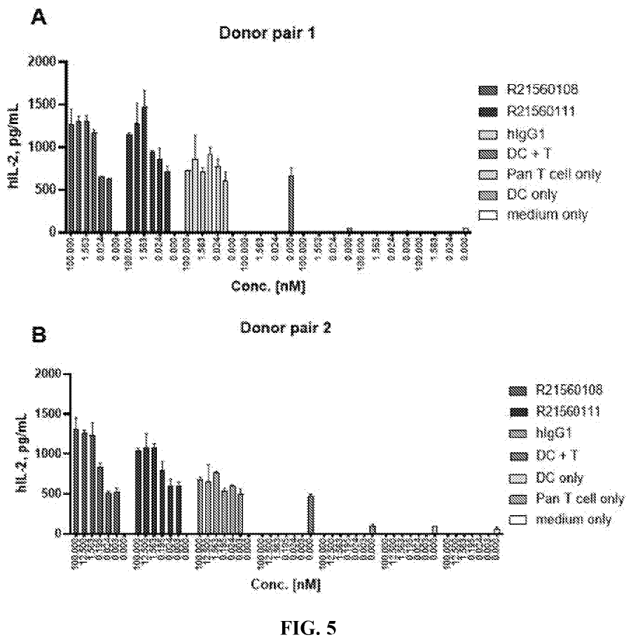

Based on these data, two of the affinity matured antibodies, R21560108 and R21560111, were selected for further study. In the mixed lymphocyte reaction (MLR) assay with two donor pairs, anti-PDL-1 antibody could dose dependently induce human IL-2 production as shown in FIG. 5.

### Example 9. In Vivo Anti-Tumor Effect of Affinity Matured Antibodies

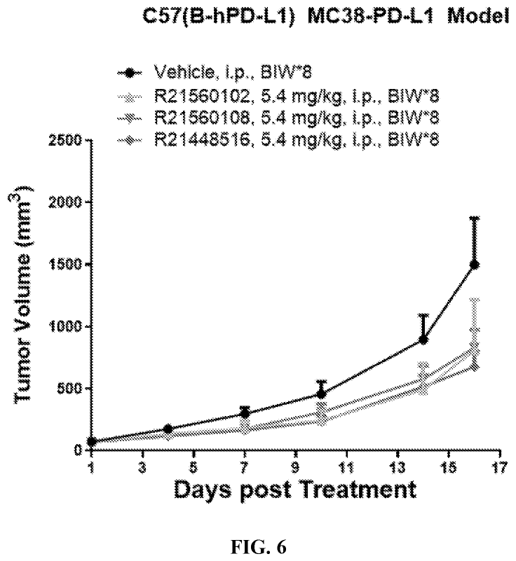

MC38-PD-L1 syngeneic model, having tumor cells expressing human PD-L1, was used to test the anti-tumor effect of the affinity matured antibodies R21560102, R21560108 and R21448516. As shown in FIG. 6, all three tested affinity-matured anti-PD-L1 antibodies showed effective tumor growth inhibition, as compared with the control group (vehicle).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A single domain antibody or a polypeptide comprising the single domain antibody, wherein the single domain antibody has binding specificity to the human PD-L1 protein and comprises a complementarity determining region 1 (CDR1), a CDR2 and a CDR3, wherein:
(1) the CDR1 comprises the amino acid sequence of SEQ ID NO:54 or 95;
the CDR2 comprises the amino acid sequence of SEQ ID NO:55 or 96; and
the CDR3 comprises the amino acid sequence of SEQ ID NO:56, 97, 98, 99, 100, 101, 102 or 103, wherein the CDR1, CDR2 and CDR3 do not comprise the sequences of SEQ ID NO:54-56, respectively; or
(2) the CDR1 comprises the amino acid sequence of SEQ ID NO:57 or 90;
the CDR2 comprises the amino acid sequence of SEQ ID NO:58 or 91; and
the CDR3 comprises the amino acid sequence of SEQ ID NO:59, 92, 93 or 94, wherein the CDR1, CDR2 and CDR3 do not comprise the sequences of SEQ ID NO:57-59, respectively.

2. The antibody or polypeptide of claim 1, wherein the CDR1, CDR2 and CDR3 comprise, respectively,
(1) SEQ ID NO:90, 58, and 59;
(2) SEQ ID NO:57, 91, and 59;
(3) SEQ ID NO:57, 58, and 92;
(4) SEQ ID NO:57, 58, and 93;
(5) SEQ ID NO:90, 58, and 92;
(6) SEQ ID NO:90, 58, and 93;
(7) SEQ ID NO:57, 91, and 92;
(8) SEQ ID NO:57, 91, and 93;
(9) SEQ ID NO:57, 58, and 94;
(10) SEQ ID NO:90, 91, and 94;
(11) SEQ ID NO:90, 91, and 92; or
(12) SEQ ID NO:90, 91, and 93.

3. The antibody or polypeptide of claim 2, which comprises an amino acid sequence having at least 85% sequence identity to SEQ ID NO:53.

4. The antibody or polypeptide of claim 2, which comprises the framework regions of SEQ ID NO:53.

5. The antibody or polypeptide of claim 2, wherein the CDR1, CDR2 and CDR3 comprise, respectively, SEQ ID NO:57, 91, and 93.

6. The antibody or polypeptide of claim 5, which comprises the amino acid sequence of SEQ ID NO: 67.

7. The antibody or polypeptide of claim 2, wherein the CDR1, CDR2 and CDR3 comprise, respectively, SEQ ID NO:90, 91, and 92.

8. The antibody or polypeptide of claim 7, which comprises the amino acid sequence of SEQ ID NO:70.

9. The antibody or polypeptide of claim 1, wherein the CDR1, CDR2 and CDR3 comprise, respectively,
(13) SEQ ID NO:95, 55, and 56;
(14) SEQ ID NO:54, 96, and 56;
(15) SEQ ID NO:54, 55, and 97;
(16) SEQ ID NO:54, 55, and 98;
(17) SEQ ID NO:54, 55, and 99;
(18) SEQ ID NO:95, 55, and 97;
(19) SEQ ID NO:95, 55, and 98;
(20) SEQ ID NO:95, 55, and 99;
(21) SEQ ID NO:54, 96, and 97;
(22) SEQ ID NO:54, 96, and 98;
(23) SEQ ID NO:54, 96, and 99;
(24) SEQ ID NO:95, 96, and 97;
(25) SEQ ID NO:95, 96, and 98;
(26) SEQ ID NO:95, 96, and 99;
(27) SEQ ID NO:95, 96, and 100;
(28) SEQ ID NO:54, 55, and 101;
(29) SEQ ID NO:54, 55, and 102; or
(30) SEQ ID NO:54, 55, and 103.

10. The antibody or polypeptide of claim 9, which comprises an amino acid sequence having at least 85% sequence identity to SEQ ID NO:42.

11. The antibody or polypeptide of claim 9, which comprises the framework regions of SEQ ID NO:42.

12. The antibody or polypeptide of claim 9, wherein the CDR1, CDR2 and CDR3 comprise, respectively, SEQ ID NO:95, 96, and 100.

13. The antibody or polypeptide of claim 12, which comprises the amino acid sequence of SEQ ID NO:86.

14-15. (canceled)

16. A multispecific antibody comprising the antibody of claim 1 and a second antibody or antigen-binding fragment having binding specificity to a target antigen that is not PD-L1.

17-18. (canceled)

19. A polynucleotide encoding the antibody or polypeptide of claim 1.

20. (canceled)

21. A cell comprising the polynucleotide of claim 19.

22. A composition comprising
(1) the antibody or polypeptide of claim 1, the multispecific antibody of any one of claims 16 to 18, the polynucleotide of claim 19, the vector of claim 20, or the cell of claim 21, and
(2) a pharmaceutically acceptable carrier.

23. A method of treating cancer in a patient in need thereof, comprising administering to the patient an effective amount of the antibody or polypeptide of claim 1.

24. (canceled)

25. The method of claim 23, wherein the cancer is a solid tumor.

26. The method of claim 23, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.


SEQUENCE LISTING (103 sequences)

SEQ ID NO: 1 - protein, 126 aa, Lama sp.
EVQLVESGGGLVQAGDSLTLSCAASGRTFSSYAMGWFRQAPGKEREFVARITWTGRSTSYADSVKGRFTISRDNAKNRVYLRMNSLKPEDTAVYYCAADLEGAMVSRRREIEYGHWGQGTQVTVSS

SEQ ID NO: 2 - protein, 122 aa, Lama sp.
EVDLVESGGGLVQAGGSLRLSCAASGGSTFAMAWLRQAPGKEREFVAAVGRSPRSPGITYYADSVKGRFTISRDNAKNTVYLQMNSLKPEDTAVYYCAAGGILGPRAHYDYWGQGTQVTVSS

SEQ ID NO: 3 - protein, 122 aa, Lama sp.
QVQLVESGGGLVQAGGSLRLSCAASGRTFSRYAMGWFRQAPGKEREFVAAISWSGGTTNYADSVKGRFTISRDNAKITVYLQMNSLKPEDTAFYYCAAGKRLTLRSSGYKYWGQGTQVTVSS

SEQ ID NO: 4 - protein, 122 aa, Lama sp.
AVQLVESGGGLVQAGGSLRLSCAASRGSTFAMAWIRQAPGKEREFVAAVGRSPRGPGITYYADSVKGRFTISRDNANNTVYLQMNSLKPEDTAVYYCAAGGILGPRAQYDYWGQGTQVTVSS

SEQ ID NO: 5 - protein, 122 aa, Lama sp.
QVQLVESGGGLVQAGSSLRLSCAASRGSTFAMAWIRQAPGKEREFVAAVGRSPRGPGITYYADSVKGRFTISRDNANNTVYLQMNSLKPEDTAVYYCAAGGILGPRAQYDYWGQGTQVTVSS

SEQ ID NO: 6 - protein, 121 aa, Lama sp.
QVQLVESGGGLVQPGGSLRLSCAASGSIFSSGTNFSDSKIDWYRQAPGKQRDWIAGIFSTGSTIYEDSVKGRFAISRDNAKNMGYLQMNSLKPEDTAVYYCRVIGRGILAWGQGTQVTVSS

SEQ ID NO: 7 - protein, 122 aa, Lama sp.
QVQLVESGGGLVQAGGSLRLSCAASRGSTFAMAWIRQAPRKEREFVPAVGRSPLGPVITYYADSVKGRFTISRDNANNTVYLQMNSLKPEDTAVDYCASCGILGPRAHYDYWGQGTHVTVSS

SEQ ID NO: 8 - protein, 126 aa, Lama sp.
EVQLVESGGGLVQAGDSLTLSCAASGRTFSSYAMGWFRQAPGKEREFVARITWSGRSTSYADSVKGRFTISRDNAKNRVYLRMNSLKPEDTAVYYCAADLEGAMVSRRREIEYGQWGQGTQVTVSS

SEQ ID NO: 9 - protein, 122 aa, Lama sp.
QVQLVESGGGLVQAGGSLRLSCAASGGSTFAMAWIRQAPGKEREFVAAVGRSPRSPGITYYADSVKGRFTISRDNANNTVYLQMNSLKPEDTAVYYCAAGGILGPRAQYDYWGQGTQVTVSS

SEQ ID NO: 10 - protein, 122 aa, Lama sp.; identical to SEQ ID NO: 5

SEQ ID NO: 11 - protein, 126 aa, Lama sp.
QVQLVESVGGLVQPGDSLRLSCLASGRTFTFRHYVMGWFRQAPGKEREFVAAISWSGSGSYYADSVKGRFTISRDNSKNMVFLQMNGLKPEDTAVYYCAADMTTRMSQASREYDYWGQGTQVTVSS

SEQ ID NO: 12 - protein, 122 aa, Lama sp.
EVQLVESGGGLVQAGGSLRLSCAASGGSTFAMAWLRQAPGKEREFVAAVGRSPRSPGITYYADSVKGRFTISRDNAKNTVWLQMNSLKPEDTAVYYCAAGGILGPRAEYDYWGQGTRVTVSS

SEQ ID NO: 13 - protein, 126 aa, Lama sp.
QVHLVESGGGLVQAGDSLTLSCAASGRTFSSYAMGWFRQAPGKEREFVARITWSGRSTSYADSVKGRFTISRDNAKNRVYLRMNSLKPEDTAVYYCAADLEGAMVSRRREIEYGHWGQGTQVTVSS

SEQ ID NO: 14 - protein, 127 aa, Lama sp.
QVQLVESGGELVQAGGSLRLSCAASGRTFSSYAMGWFRQGPGKEREFVAAISASGGRTYYADSVKGRFTISRDNAKNTVYLQMNSLKPEDTAVYYCAVAGPRIRIATITLSREYDYWGQGTQVTVSS

SEQ ID NO: 15 - protein, 115 aa, Lama sp.
AVQLVESGGGLVQAGGSLRLSCVASEIAFSVFDMGWYRQAPGKQRELAASIGHDGRINYADSVKGRFTISRDNAKNTVHLQMNTLKSEDTAVYYCNARNSFRDLWGQGTQVTVSS

SEQ ID NO: 16 - protein, 127 aa, Lama sp.
AVQLVESGGGLVQPGGSLRLSCAASGRSFSGYAMGWFRQAPGKEREFVSAISGSGRNTYYADSVKGRFTISRDNAKNTMYLQMNSLKPEDTAVYYCAVAGPAITIATMTLRGKYDYWGQGTQVTVSS

SEQ ID NO: 17 - protein, 127 aa, Lama sp.
QVHLVESGGGLVQAGDSLRLSCAASGRTFSSRAMGWFRQAPGKEREFVAAISASGSRTYYADSVKGRFTISRDNAKNTVYLQMNSLKPEDTAVYYCAAAGPRITIATMTLSREYDYWGQGTQVTVSS

SEQ ID NO: 18 - protein, 127 aa, Lama sp.
EVQLVESGGGLVQPGGSLRLSCAASGRTFSSYALGWFRQAPGKEREFVAAISASGLRTYYADSVKGRFTISRDNAKNTVYLQMNSLKPEDTAVYYCAEAGPRIRIATMTLSREYDYWGQGTLVTVSS

SEQ ID NO: 19 - protein, 127 aa, Lama sp.
QVQLVESGGGLVQAGGSLRLSCAASGRTFSSYAMGWFRQAPGKEREFATAISASGRSTYYADSVKGRFTISRDNAKNTVYLQMNSLKPEDTAVYYCAQGGPSITIRTMGSSSKYDYWGRGTQVTVSS

SEQ ID NO: 20 - protein, 127 aa, Lama sp.
QVHLVESGGGLVQAGGSLRLSCAASGRTFSSYAMGWFRQAPGKEREFVAAVSASGGRSYYVDSVKGRFTISRDNAKNTVYLQMNSLKPEDTAVYYCAAAGRSITIATMTERYKYDYWGQGTQVTVSS

SEQ ID NO: 21 - protein, 127 aa, Lama sp.
EVQLVESGGGLVQAGGSLRLSCAASGRSFSGYAMGWFRQAPGKERDFVAAISGTGGSTYYVDSVKGRFTISRDNAKNTMYLQMNSLKPEDTAVYYCAVAGPAITIATMTLRGKYDYWGQGTQVTVSS

SEQ ID NO: 22 - protein, 118 aa, Lama sp.
AVQLVESGGSLRLSCAASGRTFSSYAMGWFRQAPGKEREFVAAISGSGARTYYADSVKGRFTISRANTKNTVYLQMNSLKPEDTAVYYCAADATRIASVDVPKSWGYWGQGTQVTVSS

SEQ ID NO: 23 - protein, 126 aa, Lama sp.
QLHFVESGGGLVQAGGSLRLACAASGRTFSGYARTWFRQAPGKEREFVAAISGSGASAYYADSVKGRFTISRDNAKNTVYLQMNSLKPEDTAVYYCAADQSIRIATMRTHAAYGYWGQGTQVTVSS

SEQ ID NO: 24 - protein, 122 aa, Lama sp.
QVQLVESVGGLVQAGGSLSLSCAASGGSTFAMAWLRQAPGKEREFVAAVGRSPRGPGITNYADSVKGRFTISRDNAKNTVYLQMNSLKPEDTAVYYCAAGGILGPRAQYDYWGQGTRVTVSS

SEQ ID NO: 25 - protein, 125 aa, Lama sp.
EVHLVESGGGLVQPGGSLRLSCAHSGSIRSINVMNWYRQVPGKQRELVATITAGGSTNYADSVKGRFTISRDNALNTAALQMNSLRPEDTAVYYCHADKILTYNGVIYRAEYDVWGQGTQVTVSS

SEQ ID NO: 26 - protein, 127 aa, Lama sp.
QVQLVESGGGLVQAGGSLRLSCAASGRTFSGYAMGWFRQAPGKEREFVAAISGSGGRTYYVDSAKGRFTISRDNAKNTMYLQMNSLKPEDTAVYYCAVAGPAITIATMTLRGKYDYWGQGTQVTVSS

SEQ ID NO: 27 - protein, 125 aa, Lama sp.
QVQLVESGGGLVQPGGSLRLSCAASESIRSINVMNWYRQAPGKQRELVATITSGGTTTYADSVKGRFTSSRDNAKNTVALQMNSLRPEDTAVYYCHADKVLYYNGVIYGAEYDVWGQGTQVTVSS

SEQ ID NO: 28 - protein, 125 aa, Lama sp.
QLQLVESGGGLVQPGGSLRLSCAPSGSIRSINVMNWYRQAPGKQRDLVATITSGGSTNYADSVKGRFIISRDNAKNTVALQMNSLRPEDTAVYYCHADKVLYYNGVLYGAEYDVWGQGTQVTVSS

SEQ ID NO: 29 - protein, 131 aa, Lama sp.
EVHLVESGGGLVQAGGSLRLSCEVSGSIFSGTHFSFNTMGWYRQAPGKQRELVALGRGSRGINYADSVKGRFTFSSDNAKNTIFLQMNNLEPEDTGNYTCYVRRPSGSYAGQYYPDSSEYWGQGTQVTVSS

SEQ ID NO: 30 - protein, 111 aa, Lama sp.
QPQVVESGGGLVQAGGSLRLSCVASGSNFAFEYIAWYRQAPGKEREVVALISPQSITTYADSVKGRFTISRDNAKSTVYLQMNSLKPEDTAVYYCHDREYWGQGTQVTVSS

SEQ ID NO: 31 - protein, 125 aa, Lama sp.
QLHFVESGGGLVQPGGSLRLSCAASGSIRSINVMNYYRQAPGKQRELVATISSVGSTNYADSVKGRFTISRDNAQNTVALQMNSLRPEDTAVYYCHADKVLYYNGVMYGVESDVWGQGTQVTVSS

SEQ ID NO: 32 - protein, 128 aa, Lama sp.
QVQLVESGGGEVQPGGSLRLSCAASGPTFSRYIMGWFRQAPGKEREFVAAISRIGGITYYTDSVKGRFTISRDNAKNTVYLQMNSLEPEDTASYYCAAKSSSSSSKYTARGADAYDYWGQGTQVTVSS

SEQ ID NO: 33 - protein, 117 aa, Lama sp.
AVQLVESGGGLVQPGGSLRLSCAASRSVFSVLVMGWYRQAPGQQRELVATISNEGYSNYADSVKGRFAISRDNAKKTVYLQMNSLKPEDTAVYYCNAAWGNGRYTYWGQGTQVTVSS

SEQ ID NO: 34 - protein, 125 aa, Lama sp.
EVHLVESGGGLVQPGGSLRLSCAHSGSIRSINVMNWYRQVPGKQRELVATITAGGSTNYADSVKGRFTISRDNAKNSAALQMNSLRPEDTAVYYCHADKVLSYNGVIYRAEYDVWGQGTQVTVSS

SEQ ID NO: 35 - protein, 126 aa, Lama sp.
ELQLVESGGGLVQAGGSLRLSCAVSESIFKFPRMGWYRQGPGDQRDLVALSRSSGSTEYADFAKGRFTISRDNSKNTVYLQMNSLKPEDSGTYYCYVRRPSGSFNGRWYTDPSDDWGQGTRVTVSS

SEQ ID NO: 36 - protein, 113 aa, Lama sp.
QVQLVESGGGLVQPGGSLRLSCAASARSINGMEWYRQAPGERRELVAGITAGGSAYYTDTVKGRFTISRDNAENTGYLQMNSLSPDDTAVYYCRRQYGPNWYWGQGTQVTVSS

SEQ ID NO: 37 - protein, 126 aa, synthetic construct
EVQLVESGGGLVQPGGSLRLSCAASGFTFTFRHYVMGWVRQAPGKEREWVSAISWSGSGSYYADSVKGRFTISRDNSKNTLYLQMNSLRAEDTAVYYCAKDMTTRMSQASREYDYWGQGTLVTVSS

SEQ ID NO: 38 - protein, 126 aa, synthetic construct
EVQLVESGGGLVQPGGSLRLSCAASGFTFTFRHYVMGWVRQAPGKEREWVSAISWSGSGSYYADSVKGRFTISRDNSKNTVYLQMNSLRAEDTAVYYCAADMTTRMSQASREYDYWGQGTLVTVSS

SEQ ID NO: 39 - protein, 126 aa, synthetic construct
EVQLVESGGGLVQPGGSLRLSCAASGFTFTFRHYVMGWFRQAPGKGLEFVAAISWSGSGSYYADSVKGRFTISRDNSKNTLYLQMNSLRAEDTAVYYCAKDMTTRMSQASREYDYWGQGTLVTVSS

SEQ ID NO: 40 - protein, 126 aa, synthetic construct
EVQLVESGGGLVQPGGSLRLSCAASGFTFTFRHYVMGWFRQAPGKGLEFVAAISWSGSGSYYADSVKGRFTISRDNSKNTVYLQMNSLRAEDTAVYYCAADMTTRMSQASREYDYWGQGTLVTVSS

SEQ ID NO: 41 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTLYLQMNSLRAEDTAVYYCAKDMTTRMSQASREYDYWGQGTLVTVSS

SEQ ID NO: 42 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 43 - protein, 126 aa, synthetic construct
EVQLVESGGGLVQPGGSLRLSCAASGFTFTFRHYVMGWFRQAPGKGLEFVAAISWSGSGSYYADSVKGRFTISRDNSKNTLYLQMNSLRAEDTAVYYCAADMTTRMSQASREYDYWGQGTLVTVSS

SEQ ID NO: 44 - protein, 126 aa, synthetic construct
EVQLVESGGGLVQPGGSLRLSCAASGRTFTFRHYVMGWFRQAPGKGLEFVAAISWSGSGSYYADSVKGRFTISRDNSKNTVYLQMNSLRAEDTAVYYCAADMTTRMSQASREYDYWGQGTLVTVSS

SEQ ID NO: 45 - protein, 126 aa, synthetic construct
EVQLVESGGGLVQPGGSLRLSCAASGRTFTFRHYVMGWFRQAPGKGLEFVAAISWSGSGSYYADSVKGRFTISRDNSKNTLYLQMNSLRAEDTAVYYCAADMTTRMSQASREYDYWGQGTLVTVSS

SEQ ID NO: 46 - protein, 121 aa, synthetic construct
EVQLVESGGGLVQPGGSLRLSCAASGSIFSSGTQFSDSKIDWVRQAPGKGLEWVSGIFSTGSTIYEDSVKGRFTISRDNAKNSLYLQMNSLRAEDTAVYYCARIGRGILAWGQGTLVTVSS

SEQ ID NO: 47 - protein, 121 aa, synthetic construct
EVQLVESGGGLVQPGGSLRLSCAASGSIFSSGTQFSDSKIDWYRQAPGKQREWVSGIFSTGSTIYEDSVKGRFTISRDNAKNSLYLQMNSLRAEDTAVYYCARIGRGILAWGQGTLVTVSS

SEQ ID NO: 48 - protein, 121 aa, synthetic construct
EVQLVESGGGLVQPGGSLRLSCAASGSIFSSGTQFSDSKIDWYRQAPGKQREWVSGIFSTGSTIYEDSVKGRFTISRDNAKNSLYLQMNSLRAEDTAVYYCRVIGRGILAWGQGTLVTVSS

SEQ ID NO: 49 - protein, 121 aa, synthetic construct
EVQLVESGGGLVQPGGSLRLSCAASGSIFSSGTQFSDSKIDWYRQAPGKQREWVAGIFSTGSTIYEDSVKGRFTISRDNAKNSLYLQMNSLRAEDTAVYYCRVIGRGILAWGQGTLVTVSS

SEQ ID NO: 50 - protein, 121 aa, synthetic construct
EVQLVESGGGLVQPGGSLRLSCAASGSIFSSGTQFSDSKIDWYRQAPGKQRDWVAGIFSTGSTIYEDSVKGRFAISRDNAKNSLYLQMNSLRAEDTAVYYCRVIGRGILAWGQGTLVTVSS

SEQ ID NO: 51 - protein, 121 aa, synthetic construct
EVQLVESGGGLVQPGGSLRLSCAASGSIFSSGTQFSDSKIDWYRQAPGKQREWVAGIFSTGSTIYEDSVKGRFTISRDNAKNMLYLQMNSLRAEDTAVYYCRVIGRGILAWGQGTLVTVSS

SEQ ID NO: 52 - protein, 121 aa, synthetic construct
EVQLVESGGGLVQPGGSLRLSCAASGSIFSSGTQFSDSKIDWYRQAPGKQREWIAGIFSTGSTIYEDSVKGRFAISRDNAKNMLYLQMNSLRAEDTAVYYCRVIGRGILAWGQGTLVTVSS

SEQ ID NO: 53 - protein, 121 aa, synthetic construct
EVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGRGILAWGQGTLVTVSS

SEQ ID NO: 54 - protein, 7 aa, Lama sp.
FRHYVMG

SEQ ID NO: 55 - protein, 17 aa, Lama sp.
AISWSGSGSYYADSVKG

SEQ ID NO: 56 - protein, 15 aa, Lama sp.
DMTTRMSQASREYDY

SEQ ID NO: 57 - protein, 11 aa, Lama sp.
SGTQFSDSKID

SEQ ID NO: 58 - protein, 16 aa, Lama sp.
GIFSTGSTIYEDSVKG

SEQ ID NO: 59 - protein, 7 aa, Lama sp.
IGRGILA

SEQ ID NO: 60 - protein, 121 aa, synthetic construct
EVQLVESGGGLVQPGGSLRLSCAASGFTFSSGTQFSDSKADWYRQAPGKGLVWVAGIFSTGSTIYEDSVKGRFTISRDNAKNTGYLQMNSLRAEDTAVYYCRVIGRGILAWGQGTLVTVSS

SEQ ID NO: 61 - protein, 121 aa, synthetic construct
EVQLVESGGGLVQPGGSLRLSCAASGFTFSSGTQFSDSKIDWYRQAPGKGLVWVAGIFQTGSTIYEDSVKGRFTISRDNAKNTGYLQMNSLRAEDTAVYYCRVIGRGILAWGQGTLVTVSS

SEQ ID NO: 62 - protein, 121 aa, synthetic construct
EVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGIGILAWGQGTLVTVSS

SEQ ID NO: 63 - protein, 121 aa, synthetic construct
EVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGRGTLAWGQGTLVTVSS

SEQ ID NO: 64 - protein, 121 aa, synthetic construct
EVQLVESGGGLVQPGGSLRLSCAASGFTFSSGTQFSDSKADWYRQAPGKGLVWVAGIFSTGSTIYEDSVKGRFTISRDNAKNTGYLQMNSLRAEDTAVYYCRVIGIGILAWGQGTLVTVSS

SEQ ID NO: 65 - protein, 121 aa, synthetic construct
EVQLVESGGGLVQPGGSLRLSCAASGFTFSSGTQFSDSKADWYRQAPGKGLVWVAGIFSTGSTIYEDSVKGRFTISRDNAKNTGYLQMNSLRAEDTAVYYCRVIGRGTLAWGQGTLVTVSS

SEQ ID NO: 66 - protein, 121 aa, synthetic construct
EVQLVESGGGLVQPGGSLRLSCAASGFTFSSGTQFSDSKIDWYRQAPGKGLVWVAGIFQTGSTIYEDSVKGRFTISRDNAKNTGYLQMNSLRAEDTAVYYCRVIGIGILAWGQGTLVTVSS

SEQ ID NO: 67 - protein, 121 aa, synthetic construct
EVQLVESGGGLVQPGGSLRLSCAASGFTFSSGTQFSDSKIDWYRQAPGKGLVWVAGIFQTGSTIYEDSVKGRFTISRDNAKNTGYLQMNSLRAEDTAVYYCRVIGRGTLAWGQGTLVTVSS

SEQ ID NO: 68 - protein, 121 aa, synthetic construct
EVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGIGTLAWGQGTLVTVSS

SEQ ID NO: 69 - protein, 121 aa, synthetic construct
EVQLVESGGGLVQPGGSLRLSCAASGFTFSSGTQFSDSKADWYRQAPGKGLVWVAGIFQTGSTIYEDSVKGRFTISRDNAKNTGYLQMNSLRAEDTAVYYCRVIGIGTLAWGQGTLVTVSS

SEQ ID NO: 70 - protein, 121 aa, synthetic construct
EVQLVESGGGLVQPGGSLRLSCAASGFTFSSGTQFSDSKADWYRQAPGKGLVWVAGIFQTGSTIYEDSVKGRFTISRDNAKNTGYLQMNSLRAEDTAVYYCRVIGIGILAWGQGTLVTVSS

SEQ ID NO: 71 - protein, 121 aa, synthetic construct
EVQLVESGGGLVQPGGSLRLSCAASGFTFSSGTQFSDSKADWYRQAPGKGLVWVAGIFQTGSTIYEDSVKGRFTISRDNAKNTGYLQMNSLRAEDTAVYYCRVIGRGTLAWGQGTLVTVSS

SEQ ID NO: 72 - protein, 126 aa, synthetic construct
EVQLVESGGGLVQPGGSLRLSCAASGFTFTFYHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 73 - protein, 126 aa, synthetic construct
EVQLVESGGGLVQPGGSLRLSCAASGFTFTFRHYVMGWFRQAPGKEMEFVADISWSGSGSYYADSVKGRFTISRDNSKNTVYLQMNSLRAEDTAVYYCAADMTTRMSQASREYDYWGQGTLVTVSS

SEQ ID NO: 74 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTEMSQASREYDYWGQGTLVTVSS

SEQ ID NO: 75 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQDSREYDYWGQGTLVTVSS

SEQ ID NO: 76 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASDEYDYWGQGTLVTVSS

SEQ ID NO: 77 - protein, 126 aa, synthetic construct
EVQLVESGGGLVQPGGSLRLSCAASGFTFTFYHYVMGWFRQAPGKEREFVAAISWSGSGSYYADSVKGRFTISRDNSKNTVYLQMNSLRAEDTAVYYCAADMTTEMSQASREYDYWGQGTLVTVSS

SEQ ID NO: 78 - protein, 126 aa, synthetic construct
EVQLVESGGGLVQPGGSLRLSCAASGFTFTFYHYVMGWFRQAPGKEREFVAAISWSGSGSYYADSVKGRFTISRDNSKNTVYLQMNSLRAEDTAVYYCAADMTTRMSQDSREYDYWGQGTLVTVSS

SEQ ID NO: 79 - protein, 126 aa, synthetic construct
EVQLVESGGGLVQPGGSLRLSCAASGFTFTFYHYVMGWFRQAPGKEREFVAAISWSGSGSYYADSVKGRFTISRDNSKNTVYLQMNSLRAEDTAVYYCAADMTTRMSQASDEYDYWGQGTLVTVSS

SEQ ID NO: 80 - protein, 126 aa, synthetic construct
EVQLVESGGGLVQPGGSLRLSCAASGFTFTFRHYVMGWFRQAPGKEMEFVADISWSGSGSYYADSVKGRFTISRDNSKNTVYLQMNSLRAEDTAVYYCAADMTTEMSQASREYDYWGQGTLVTVSS

SEQ ID NO: 81 - protein, 126 aa, synthetic construct
EVQLVESGGGLVQPGGSLRLSCAASGFTFTFRHYVMGWFRQAPGKEMEFVADISWSGSGSYYADSVKGRFTISRDNSKNTVYLQMNSLRAEDTAVYYCAADMTTRMSQDSREYDYWGQGTLVTVSS

SEQ ID NO: 82 - protein, 126 aa, synthetic construct
EVQLVESGGGLVQPGGSLRLSCAASGFTFTFRHYVMGWFRQAPGKEMEFVADISWSGSGSYYADSVKGRFTISRDNSKNTVYLQMNSLRAEDTAVYYCAADMTTRMSQASDEYDYWGQGTLVTVSS

SEQ ID NO: 83 - protein, 126 aa, synthetic construct
EVQLVESGGGLVQPGGSLRLSCAASGFTFTFYHYVMGWFRQAPGKEREFVADISWSGSGSYYADSVKGRFTISRDNSKNTVYLQMNSLRAEDTAVYYCAADMTTEMSQASREYDYWGQGTLVTVSS

SEQ ID NO: 84 - protein, 126 aa, synthetic construct
EVQLVESGGGLVQPGGSLRLSCAASGFTFTFYHYVMGWFRQAPGKEREFVADISWSGSGSYYADSVKGRFTISRDNSKNTVYLQMNSLRAEDTAVYYCAADMTTRMSQDSREYDYWGQGTLVTVSS

SEQ ID NO: 85 - protein, 126 aa, synthetic construct
EVQLVESGGGLVQPGGSLRLSCAASGFTFTFYHYVMGWFRQAPGKEREFVADISWSGSGSYYADSVKGRFTISRDNSKNTVYLQMNSLRAEDTAVYYCAADMTTRMSQASDEYDYWGQGTLVTVSS

SEQ ID NO: 86 - protein, 126 aa, synthetic construct
EVQLVESGGGLVQPGGSLRLSCAASGFTFTFYHYVMGWFRQAPGKEREFVADISWSGSGSYYADSVKGRFTISRDNSKNTVYLQMNSLRAEDTAVYYCAADMTTEMSQDSDEYDYWGQGTLVTVSS

SEQ ID NO: 87 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTEMSQDSREYDYWGQGTLVTVSS

SEQ ID NO: 88 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTEMSQASDEYDYWGQGTLVTVSS

SEQ ID NO: 89 - protein, 126 aa, synthetic construct
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQDSDEYDYWGQGTLVTVSS

SEQ ID NO: 90 - protein, 11 aa, synthetic construct
SGTQFSDSKAD

SEQ ID NO: 91 - protein, 16 aa, synthetic construct
GIFQTGSTIYEDSVKG

SEQ ID NO: 92 - protein, 7 aa, synthetic construct
IGIGILA

SEQ ID NO: 93 - protein, 7 aa, synthetic construct
IGRGTLA

SEQ ID NO: 94 - protein, 7 aa, synthetic construct
IGIGTLA

SEQ ID NO: 95 - protein, 7 aa, synthetic construct
FYHYVMG

SEQ ID NO: 96 - protein, 17 aa, synthetic construct
DISWSGSGSYYADSVKG

SEQ ID NO: 97 - protein, 15 aa, synthetic construct
DMTTEMSQASREYDY

SEQ ID NO: 98 - protein, 15 aa, synthetic construct
DMTTRMSQDSREYDY

SEQ ID NO: 99 - protein, 15 aa, synthetic construct
DMTTRMSQASDEYDY

SEQ ID NO: 100 - protein, 15 aa, synthetic construct
DMTTEMSQDSDEYDY

SEQ ID NO: 101 - protein, 15 aa, synthetic construct
DMTTEMSQDSREYDY

SEQ ID NO: 102 - protein, 15 aa, synthetic construct
DMTTEMSQASDEYDY

SEQ ID NO: 103 - protein, 15 aa, synthetic construct
DMTTRMSQDSDEYDY